(19) United States
(12) Patent Application Publication
(10) Pub. No.: US 2012/0214874 A1
(43) Pub. Date: Aug. 23, 2012

(54) PHARMACEUTICAL FORMULATIONS AND METHODS OF USE

(75) Inventors: Nadir Buyuktimkin, San Diego, CA (US); Servet Buyuktimkin, San Diego, CA (US); Jagat Singh, Toronto (CA); John M. Newsam, La Jolla, CA (US); Dominic King-Smith, San Diego, CA (US); Edward Kisak, San Diego, CA (US); Bradley S. Galer, West Chester, PA (US); Tejas Desai, Mississauga (CA); Christopher Riley, Maryville, MO (US)

(73) Assignee: Nuvo Research Inc., Mississauga, ON (CA)

(21) Appl. No.: 13/392,498

(22) PCT Filed: Aug. 26, 2010
(86) PCT No.: PCT/US10/46875

§ 371 Date: May 4, 2012

Related U.S. Application Data
(60) Provisional application No. 61/237,252, filed on Aug. 26, 2009.

(51) Int. Cl. A61K 31/167; A61K 47/22; A61P 23/00; A61K 47/14

(52) U.S. Cl. 514/626; 514/785

(57) ABSTRACT

In one embodiment, the present invention provides a topical composition including a topically acting anesthetic active ingredient; an ester; a non-ionic surfactant; a polar solvent; water, and optionally a buffer, a pH adjusting agent or an anti-oxidant. The compositions are useful for alleviating pain especially associated with acute herpes zoster.

### BACKGROUND OF THE INVENTION

Acute herpes zoster (“AHZ”) is commonly known as “shingles.” Each year, it afflicts approximately 1 million Americans (see, Weaver B A., J Am Osteopath Assoc. 2007 March; 107(3 Suppl 1):S2-7; Website of Center for Disease Control) and 1.8 million Europeans within the 25 EU countries (see, Johnson R W, Rice A S. Pain. 2007 March; 128(1-2):3-5. Epub 2006 Dec. 11). The vast majority of these patients are middle-aged or elderly, with at least half over 50 years of age. The major risk factor for developing AHZ is age (over 50 years old), although compromised immune function due either to immune disorder or medication such as that used in chemotherapy can also increase risk.

Local anesthetics are frequently used topically to provide anesthesia on intact skin, for example prior to minor dermatological procedures or superficial venous access. Topical lidocaine is also available in an adhesive patch format under the trade name Lidoderm® for the relief of pain associated with postherpetic neuralgia, a neuropathic pain condition that a small percentage of AHZ patients will develop after healing of the rash associated with AHZ. However, at present there are no topical drugs approved in the United States indicated for the treatment of the pain associated with acute herpes zoster and current FDA-approved products have characteristics that make them unsuitable for treating this condition. For example removal of a Lidoderm® patch applied to the rash of an AHZ patient would likely be a painful experience for the patient given the skin lesions that form with AHZ, the allodynia that usually accompanies the condition, and, moreover, damage to the rash area caused by removing the patch might impede healing. In addition, covering the open skin lesion with the patch may provide a positive environment for bacteria and fungal growth, increasing the risk for infection. Indeed the FDA prescribing information for Lidoderm® specifically emphasizes that the drug should only be applied to intact skin.

U.S. Patent Publication No. 2006/0110415 to Gupta discloses a topical delivery system for cosmetic and pharmaceutical compositions comprising a skin penetration enhancing agent such as a ester of an hydroxyl acid, and a cosmetic and pharmaceutical agent. This is another example of a product that is not suitable to treat AHZ.

Given the high incidence rate of AHZ and the excellent safety profile that can be achieved with topical drugs, there is a strong unmet need for a topical treatment for the pain associated with AHZ. The present invention provides topical formulations for relief of pain associated with acute herpes zoster.

### BRIEF SUMMARY OF THE INVENTION

Acute herpes zoster is associated with skin rashes and lesions, and thus a non-stinging and low-irritancy topical formulation is strongly preferred for treatment. As such, in one embodiment, the present invention provides a topical composition, comprising, consisting essentially of, or consisting of:

a) a topically acting anesthetic active ingredient;

b) an ester selected from the group consisting of a citric acid ester and ethyl acetate;

c) a non-ionic surfactant;

d) a polar solvent; and

e) water.

The composition is useful for the management of pain associated with an acute herpes zoster infection. The composition may be made sterile or bacteriostatic for safe application to skin that is compromised by AHZ.

In certain aspects, the composition is sprayable or foamable, and as such, it is easy to apply to a wide area of the skin, or alternatively, a more localized, limited area of skin. Further, it can be applied without a need for the user to touch the skin to apply or spread the formulation, avoiding discomfort associated with allodynia (e.g., pain because of rubbing).

In a preferred aspect, the ester is a citric acid ester, such as triethyl citrate. Preferably, the ester is triethyl citrate.

In certain aspects, the composition is homogeneous. In another aspect, the composition is a microemulsion. Preferably, the microemulsion appears homogeneous to the eye.

In certain preferred aspects, the formulation optionally includes a buffer, a pH-adjusting agent, or an anti-oxidant.

In another embodiment, the present invention provides a method for alleviating pain, comprising: applying to an affected area a composition, comprising, consisting essentially of, or consisting of:

a) a topically acting anesthetic active ingredient;

b) an ester selected from the group consisting of a citric acid ester and ethyl acetate;

c) a non-ionic surfactant;

d) a polar solvent; and

e) water, thereby alleviating pain.

In certain preferred aspects, the composition disclosed herein comprises the topically acting anesthetic active agent lidocaine, and the composition approximates the lidocaine penetration and pharmacokinetics obtained with a Lidoderm® patch.

These and other objects, embodiments, and advantages will become more apparent when read with the figures and detailed description which follow.

### BRIEF DESCRIPTION OF THE DRAWINGS

FIG. 1 illustrates a schematic representation of an accumulated dose of lidocaine, a lidocaine salt or a combination using formulation embodiments of the present invention.

FIG. 2 illustrates a schematic representation of an accumulated dose of lidocaine, a lidocaine salt or a combination using an embodiment of the present invention compared to Lidoderm®.

FIG. 3 illustrates a schematic representation of an accumulated dose of lidocaine, a lidocaine salt or a combination using formulation embodiments of the present invention.

FIG. 4 illustrates a schematic representation of an accumulated dose of lidocaine, a lidocaine salt or a combination using formulation embodiments of the present invention.

FIG. 5 illustrates a schematic representation of an accumulated dose of lidocaine, a lidocaine salt or a combination using formulation embodiments of the present invention.

FIG. 6 illustrates a schematic representation of an accumulated dose of lidocaine, a lidocaine salt or a combination using an embodiment of the present invention compared to a commercial medicament.

FIG. 7 illustrates a schematic representation of an accumulated dose of lidocaine, a lidocaine salt or a combination using formulation embodiments of the present invention.

FIG. 8 illustrates a schematic representation of an accumulated dose of lidocaine, a lidocaine salt or a combination using an embodiment of the present invention compared to a commercial medicament.

FIG. 9 illustrates a schematic representation of an accumulated dose of lidocaine, a lidocaine salt or a combination using formulation embodiments of the present invention.

FIG. 10 illustrates a schematic representation of an accumulated dose of lidocaine, a lidocaine salt or a combination using formulation embodiments of the present invention.

FIG. 11 illustrates a schematic representation of an accumulated dose of lidocaine, a lidocaine salt or a combination using formulation embodiments of the present invention.

FIG. 12 illustrates a schematic representation of an accumulated dose of lidocaine, a lidocaine salt or a combination using formulation embodiments of the present invention.

FIG. 13 illustrates a schematic representation of the skin retention of lidocaine, a lidocaine salt or a combination after 24 h using formulation embodiments of the present invention.

FIG. 14 illustrates a schematic representation of an accumulated dose of lidocaine, a lidocaine salt or a combination using formulation embodiments of the present invention

FIG. 15 illustrates a schematic representation of an accumulated dose of lidocaine, a lidocaine salt or a combination using an embodiment of the present invention compared to Lidoderm®.

FIG. 16 illustrates a schematic representation of an accumulated dose of lidocaine, a lidocaine salt or a combination using formulation embodiments of the present invention.

FIG. 17 illustrates a schematic representation of an accumulated dose of lidocaine, a lidocaine salt or a combination using formulation embodiments of the present invention.

FIG. 18 illustrates a schematic representation of an accumulated dose of lidocaine, a lidocaine salt or a combination using formulation embodiments of the present invention.

FIG. 19 illustrates a schematic representation of an accumulated dose of lidocaine, a lidocaine salt or a combination using formulation embodiments of the present invention.

FIG. 20 illustrates a schematic representation of an accumulated dose of lidocaine, a lidocaine salt or a combination using formulation embodiments of the present invention.

FIG. 21 illustrates a schematic representation of an accumulated dose of lidocaine, a lidocaine salt or a combination using formulation embodiments of the present invention.

FIG. 22 illustrates a tabular summary of stability data using a formulation embodiment of the present invention.

FIG. 23 illustrates a schematic representation of an accumulated dose of lidocaine, a lidocaine salt or a combination using formulation embodiments of the present invention.

FIGS. 24 A-C illustrate an effect of buffer concentration on a formulation component in an embodiment of the present invention.

FIGS. 25 A-B illustrate an effect of buffer concentration on a formulation component in an embodiment of the present invention.

FIG. 26 illustrates permeation results through abraded cadaver skin. The results are for formulations of the present invention compared to a commercial medicament.

FIGS. 27 A-B illustrate permeation results through intact and abraded porcine skin The results are for formulations of the present invention compared to a commercial medicament.

### DETAILED DESCRIPTION OF THE INVENTION

### I. Definitions

The terms “a,” “an,” or “the” as used herein not only includes aspects with one member, but also includes aspects with more than one member. For example, an embodiment including “a topically acting anesthetic ingredient and a surfactant” should be understood to present certain aspects with two or more topically acting anesthetic ingredients, two or more surfactants, or both.

In compositions consisting of, consisting essentially of, or comprising a “first” and a “second” component, the second component as used herein is chemically different from the first component (e.g., a mixture comprising a first liquid such as triethyl citrate and a second liquid such as water).

The term “about” as used herein, includes a close, but imprecise quantity of a value. For example, in certain instances the term about includes as much as 5%, 6%, 7%, 8%, 9%, or 10% higher, or as much as 5%, 6%, 7%, 8%, 9%, or 10% lower than the explicit value given. For example, “about 10” includes the range of values from 9.5 to 10.5.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

In general, the “error bars” on the graphs represent the standard error of the mean value, whereas the top of the bar represents a single data value, which is the mean value of the distribution of data values.

The term “transdermal” is used herein to include a process that occurs through the skin. The terms “transdermal” and “percutaneous” are used interchangeably throughout this specification.

The term “finite dosing” is used herein to generally include application of a limited reservoir of a formulation containing an active agent. The active agent in the reservoir is depleted with time leading to a tapering-off of the active absorption rate after a maximum absorption rate is reached.

The term “infinite dosing” is used herein to generally include an application of a large reservoir of a formulation containing an active agent. The active agent in the reservoir is not significantly depleted with time, at least over the time frame intended for the reservoir to be in contact with the skin, thereby providing a long-term, continuous, steady-state absorption of the active.

“Lower alkanol” as used herein includes straight- or branched-chain alkyl alcohols of 1 to about 6 carbon atoms. Representative lower alkanols include methanol, ethanol, n-propanol, isopropanol, n-butanol, t-butanol, n-pentanol, 3-pentanol, and the like.

A “solution” as used herein includes a homogeneous mixture composed of two or more substances. A solution can be formed by dissolving a solute in another substance, known as a solvent.

A “microemulsion” as used herein is a mixture of two or more substantially immiscible liquids, wherein the first liquid comprises the dispersed phase and the other liquid comprises the continuous phase. In one aspect, the microemulsion comprises an oil-in-water (o/w) microemulsion wherein the continuous phase comprises water and the dispersed phase comprises oil. In another aspect, the microemulsion comprises a water-in-oil (w/o) microemulsion, wherein the continuous phase comprises oil and the dispersed phase comprises water. In certain aspects, the microemulsion may appear homogeneous to the eye as the particles of the dispersed phase are smaller than the wavelength of visible light (about 400 to about 700 nm).

Mixtures of substantially immiscible liquids may possess a degree of solubility, so that at low, but detectable concentrations of a first liquid in a second liquid, the mixture may be a solution. In certain aspects, the term “microemulsion” as used herein is intended to include compositions in which the mixture of substantially immiscible liquids comprises a low, but detectable concentration of a first liquid (e.g., triethyl citrate) in a second liquid (e.g., water).

The term “non-irritating” as used herein includes compositions for which any inflammatory skin reaction at the application site is imperceptible or sufficiently mild as to not preclude topical or transdermal administration. An irritancy study can be conducted to assess whether the novel topical formulations described herein cause irritation of the skin. See, e.g., Example 25.

The term “non-stinging” as used herein includes compositions that are substantially without the perception of stinging, pain, or of a distinct discomfort to the user when applied.

A stinging test can be used to assess whether the novel topical formulations described herein produce a sensory perception of stinging. See, e.g., Example 24.

The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a formulation pH that is between 9 and 10 or between 7 and 8).

Generally, when a percentage range is taught, it incorporates all full or partial percentages in-between (i.e., within the bounds of the range). For example, a percentage range of 15 to 25% would also teach the specific values of 17.36% and 21%. A percentage range of about 13% to 17% would also teach the specific values of 12.97%, 16%, and 17.1%.

The term “spray” is used herein to include a jet composed of finely divided liquid.

The term “spray-pumpable” is used herein to include formulations that are liquid at 15-30° C. under normal atmospheric pressure, that may be dispensed as a spray from a hand-held spray pump dispenser by applying normal finger pressure to the portion of the spray pump assembly designed to be activated by finger pressure. (See, e.g., U.S. Pat. Nos. 3,159,316, 4,034,900, and 4,050,860, which show different spray pump dispensers.) The hand-held spray pump dispenser used to dispense (spray) a composition of this invention typically contains the composition at atmospheric pressure and it is only when finger pressure is applied that the spray pump mechanism temporarily pressurizes the composition to cause a portion of it to leave the dispenser as a spray. The pressure in the mechanism soon returns to atmospheric after the small portion of composition has been dispensed. Such a hand-held spray pump dispenser is considered to be a non-pressurized dispenser. In certain preferred embodiments of this invention, a hand-held spray pump dispenser (i.e., a non-pressurized dispenser) can be used in its normal manner to dispense the composition of this invention.

The phrase “substantially free” of a lower alcohol is used herein to include “essentially free” of a lower alkanol. Such embodiments may include trace amounts or de minimus amounts of a lower alkanol.

The term “topical composition” is used herein to generally include a formulation that can be applied to skin or a mucosa. Topical formulations may, for example, be used to confer therapeutic benefit to a patient or cosmetic benefits to a consumer. Topical compositions can be used for both topical and transdermal administration of substances. In a preferred embodiment, the topical composition of the present invention provides a therapeutic benefit to a patient.

The term “topical administration” is used herein to generally include the delivery of a substance, such as a therapeutically active agent, into the skin or to a localized region of the body via the skin.

The term “transdermal administration” is used herein to generally include administration through the skin. Transdermal administration is often applied where systemic delivery of an active is desired, although it may also be useful for delivering an active to tissues underlying the skin with minimal systemic absorption.

### II. Formulations

In one embodiment, the present invention provides a topical composition, comprising, consisting essentially of, or consisting of:
a) a topically acting anesthetic active ingredient;
b) an ester selected from the group consisting of a citric acid ester and ethyl acetate;
c) a non-ionic surfactant;
d) a polar solvent; and
e) water.

In one aspect, the composition of the present invention is a microemulsion. Preferably, the microemulsion appears homogeneous to the eye.

In one aspect, the composition is homogeneous.

In one aspect, the topically acting anesthetic active ingredient includes, but is not limited to, an ingredient from the group tetracaine, lidocaine, prilocalne, benzocaine, bupivacaine, mepivacaine, dibucaine, etidocaine, butacaine, cyclomethycaine, hexylcaine, proparacaine, lopivacaine and pharmaceutically acceptable salts thereof. In certain preferred aspects, the active ingredient is lidocaine hydrochloride or lidocaine base. Lidocaine hydrochloride is especially preferred. In another preferred embodiment, the anesthetic active is a combination of lidocaine and lidocaine hydrochloride in a ratio from about 10:1 to about 1:10, such as 1:1. When the anesthetic contains a basic functionality, it may be present in the form of an acid addition salt or as the free base. Preferred salts are the hydrochloride, hydrobromide, acetate, citrate, carbonate or sulfate salts. In one embodiment, the active is lidocaine hydrochloride monohydrate.

In certain aspects, the amount of topically acting anesthetic active is effective to achieve analgesia without anesthesia i.e., a subanesthetic effective amount. It is believed that the dose to achieve analgesia is below the dose to achieve anesthesia (i.e., a lower dose). The dose maintains an effective amount of, for example, lidocaine intradermally, for an extended period of time to maintain extended relief from pain. In certain aspects, the topically acting anesthetic active ingredient is in amount of about 0.1% to about 20% weight by weight (“w/w”). In another embodiment, the topically acting anesthetic active ingredient is in an amount of about 5% to about 20% w/w, such as about 6% to about 13% w/w. In another embodiment, the amount is about 1% to about 10% w/w such as for example, 1, 2, 3, 4, 5, 6, 7, 8, 9, or 10% w/w, and all fractions in-between. In other aspects, the amount of topically acting anesthetic active is about 5% or about 10% w/w, such as 5% or 10%.

In certain aspects, the composition of the present invention comprises an ester in an amount of about 0.01% to about 20% w/w. In one embodiment, the amount of the ester is about 0.01% to about 5% w/w, such as 0.01, 0.1, 0.5, 1, 2, 3, 4, or 5% w/w, and all fractions in-between. In another embodiment, the amount of the ester is about 5% to about 10% w/w such as 5, 6, 7, 8, 9, or 10% w/w, and all fractions in-between. In other aspects, the amount of ester is about 15% w/w to about 20% w/w, or 15% w/w or 20% w/w.

In a preferred aspect, the amount of the ester is less than about 20%, 15%, 10%, 5%, 1%, or fractions in-between. In certain aspects, inclusion of a high percentage of the ester component necessitates the inclusion of a larger amount of a surfactant component to produce a homogeneous composition. In certain instances, a larger amount of surfactant component may provoke skin irritation or a stinging sensation upon application, which is disadvantageous for effective topical analgesia and especially disadvantageous for the treatment of acute herpes zoster.

In certain aspects, the ester is a carboxylic acid ester selected from the group of an acetic acid ester, a propanoic acid ester, a butyric acid ester, a citric acid ester, a tartaric acid ester, an atopic acid ester, a malic acid ester, a maleic acid ester and combinations thereof. In one aspect, the ester is a citric acid ester, such as triethyl citrate.

In certain aspects, the citric acid ester of the present invention is an esterification product of citric acid and an alcohol or acid. Suitable alcohols include, but are not limited to, methanol, ethanol, propanol, isopropanol, butanol, and polyols, such as glycerol, propylene glycol, butylene glycol and dipropylene glycol, and combinations thereof. As citric acid possesses an alcohol functionality, it is possible to esterify the same with an acid (e.g., acetic acid) or other carboxylic acid.

In a preferred aspect, the citric acid ester is selected from the group consisting of triethyl citrate, acetyl triethyl citrate, tributyl citrate, acetyl tributyl citrate and a combination thereof. In one embodiment, the citric acid ester is triethyl citrate. In an alternative embodiment, the ester is ethyl acetate.

In certain instances, heating of the formulation may produce reaction of the ester with an amine component in the anesthetic active (e.g., amidation or base-induced decomposition). In a preferred aspect, the topical composition of the present invention is prepared without heating. Alternatively, the topical composition is prepared with heating to increase solubility, but heating is mild or too brief to produce significant side reactions.

In certain instances, the present invention provides a composition with a non-ionic surfactant, wherein the non-ionic surfactant is in an amount of about 2% to about 10% w/w such as about 2%, 3%, 4%, 5%, 6%, 7%, 8%, 9% or 10% w/w, and fractions in-between. In a preferred aspect, the non-ionic surfactant is in an amount of less than about 10%, 9%, 8%, 7%, 6%, 5%, 4%, or 3%.

A variety of non-ionic surfactants are suitable for the present invention. Such non-ionic surfactants include, for example, a sorbitan fatty acid ester, a sorbitol fatty acid ester, a polyoxyethylene sorbitan fatty acid ester, polysorbate, a polyoxyethylene fatty acid ester, a polyoxyethylene alkyl ether, a polyoxyethylene hydrogenated castor oil derivative (PEGCastor oil), a polyoxyethylene polyoxypropylene alkyl ether, and a combination thereof.

Suitable non-ionic surfactants include, for example, sorbitan monolaurate, sorbitan monopalmitate, sorbitan monostearate, sorbitan sesquistearate, polyoxyethylene sorbitan monolaurate, polyoxyethylene monopalmitate, polyoxyethylene sorbitan monostearate, polyoxyethylene sorbitan tristearate, polyoxyethylene sorbitan monooleate, polyoxyethylene sorbitan trioleate, polyoxyethylene sorbitol monolaurate, polyoxyethylene sorbitol hexastearate, polyoxyethylene sorbitol tetraoleate, polyoxyethylene lauryl ester, polyoxyethylene stearyl ester, polyoxyethylene oleyl ester, polyoxyethylene lauryl ether, polyoxyethylene cetyl ether, polyoxyethylene stearyl ether, polyoxyethylene oleyl ether, polyoxyethylene hexadecyl ether, propylene glycol monostearate, polyoxypropylene, polyoxyethylene cetyl ether and a combination thereof.

In a preferred aspect, the non-ionic surfactants include polyoxyethylene (20) sorbitan monolaurate (Tween 20™) and polyoxyethylene (20) sorbitan monooleate (Tween 80™).

In certain instances, the present invention provides a composition having a polar solvent. Typically, the polar solvent is present in an amount of about 5% to about 25% w/w. For example, in certain instances, the polar solvent is present in an amount of 5%, 6%, 7%, 8%, 9%, 10% 11%, 12%, 13%, 14%, 15%, 16%, 17%, 18%, 19%, 20%, 21%, 22%, 23%, 24%, or 25% w/w.

In certain preferred aspects, the polar “solvent” is a mixture of polar solvents. In one embodiment, the invention provides a composition including a first polar solvent and a second polar solvent. The first polar solvent is present in an amount of about 0.5, 1.0, 1.5, 2.0, 2.5, 3.0, 3.5, 4.0, 4.5, 5.0, 6, 7, 8, 9, 10, 11, 12, 13, 14, or 15%. Preferably, the first polar solvent is panthenol. The second polar solvent is present in an amount of about 1.0, 2.0, 3.0, 4.0, 5.0, 6, 7, 8, 8.5, 9, 10, 11, 12, 13, 14, 15, 16, 17, 18, 19, 20, 21, 22, 23, 24, or 25%. Preferably, the second polar solvent is diethylene glycol monoethyl ether (Transcutol®),

In certain aspects, the polar solvent is a diol, a triol, a polyol, diethylene glycol monoethyl ether (Transcutol®), a low-weight poly(ethylene glycol) (“PEG”), or 2,4-dihydroxy-N-(3-hydroxypropyl)-3,3-dimethyl-butanamide (panthenol).

Suitable diols include, but are not limited to, propylene glycol, butanediol, butynediol, pentanediol, hexanediol, octanediol, neopentyl glycol, 2-methyl-1,3-propanediol, diethylene glycol, triethylene glycol, tetraethylene glycol, dipropylene glycol, dibutylene glycol, propylene glycol, and a combination thereof.

Suitable triols include, but are not limited to, glycerine, 1,2,6-hexanetriol and a combination thereof. Those of skill in the art will know of other triols suitable for the present invention.

In other aspects, the polar solvent is a low-weight poly(ethylene glycol) (“PEG”). In certain aspects, the PEG is PEG 200, PEG 300, PEG 400, PEG 540, PEG 600, PEG 800, PEG 900, PEG 1000, PEG 1450, PEG 1540 and a combination thereof. In an especially preferred aspect, the low-weight PEG is PEG 300.

In certain aspects, the polar solvent is diethylene glycol monoethyl ether (Transcutol®). In other aspects, the polar solvent is panthenol. Although racemic panthenol is a water-soluble solid at room temperature, it is understood to constitute a “polar solvent” as described herein.

In certain aspects, the polar solvent is racemic (e.g., racemic panthenol). Alternatively, the polar solvent is enantiomerically enriched or is substantially a single enantiomer (e.g., (R)-panthenol, also termed D-panthenol or dexpanthenol). For example, the term panthenol as used herein can refer to racemic panthenol, panthenol enantiomerically enriched in D- or L-panthenol, or substantially one enantiomer of panthenol, unless the context precludes such a broad interpretation of the term.

In certain embodiments, the inventive compositions of the present invention are substantially free or essentially free of a lower alkanol. Such embodiments may include trace amounts of a lower alkanol. In other aspects, the composition includes a lower alkanol, such as methanol, ethanol, propanol, isopropanol, butanol, isobutanol and the like or mixtures thereof. In certain embodiments, the alkanol is a C₁-C₄ alkanol, a C₂-C₃ alkanol, or ethanol. Preferably, the lower alkanol is used at about 0-5% w/w, such as up to 5% w/w, for example, 0, 1, 2, 3, 4, or 5% w/w, and all fractions in-between. In another embodiment, if present, the lower alkanol is used at an amount of up to 3% w/w.

The compositions of the present invention preferably contain water. In certain aspects, water is present from about 30% to about 80% w/w. Preferably, water is present from about 50% to about 70% w/w, such as 55%, 60%, 65%, or 70%.

In certain embodiments, the inventive compositions include a water component of more than about 40%, or more than about 50%, such as 60%, 70%, 80% or 90%. In certain instances, the amount of water is about 40% to about 70%, such as 45%, 50%, 55%, 60%, 65%, 70% and all numbers in-between. Water amounts such as 48%, 49%, 50% 51%, 52%, 53%, 54%, 55%, 56%, 57%, 58%, 59%, 60% 61%, 62%, 63%, 64%, 65%, 66%, 67%, 68%, 69% or 70% can be used. In an alternative embodiment, the water is added quantum sufficiat (qs) or as much as suffices.

In certain preferred aspects, the compositions of the invention optionally include a buffer, a pH-adjusting agent, or an anti-oxidant. The topical formulations of the present invention may, for example, comprise a pH-adjusting agent. In one particular embodiment, the pH adjusting agent is a base. Suitable pH adjusting bases include amines, such as diethanolamine, triethanolamine, or aminopropanol; bicarbonates; carbonates; and hydroxides, such as ammonium hydroxide, alkali or alkaline earth metal hydroxide, or transition metal hydroxides. Alternatively, the pH adjusting agent can also be an acid, an acid salt, or mixtures thereof.

Preferably, the pH-adjusting agent is sodium hydroxide, hydrochloric acid, or a combination of both, and is present in an amount sufficient to adjust the pH of the composition to between about pH 4.0 to about 8.5, more preferably to between about pH 5.5 to about 7.0, such as 6.0 or 6.5. Even more preferably, the pH is adjusted to about 4.0, 4.2, 4.4, 4.6, 4.8, 5.0, 5.2, 5.4, 5.6, 5.8, 6.0, 6.2, 6.3, 6.4, 6.6, 6.8, 7.0, 7.2, 7.4, 7.6, 7.8, 8.0, 8.4, 8.5, or any fraction in-between.

In certain preferred aspects, a small amount of acid or base is included in the formulation. Non-limiting examples of amounts of acid or base that may be included in the formulation are about 0.000001%, 0.00001%, 0.0001%, 0.001%, 0.0012%, 0.01%, 0.012%, 0.1%, or 1.0%. Preferably, this amount is about 0.0001%, 0.0002%, 0.0003%, 0.0004%, 0.0005%, 0.0006%, 0.0007%, 0.0008%, 0.0009%, 0.0010%, 0.0011%, 0.0012%, 0.0015%, 0.0016%, 0.0017%, 0.0018%, 0.0019%, 0.002%, 0.003%, 0.004%, 0.005%, 0.006%, 0.007%, 0.008%, 0.009%, 0.01%, 0.012%, or 0.02%. More preferably, this amount is about 0.001%, 0.002%, 0.003%, 0.004%, 0.005%, 0.006%, 0.007%, 0.008%, 0.009%, 0.010%, 0.011%, 0.012%, 0.015%, 0.016%, 0.017%, 0.018%, 0.019%, 0.02%, 0.03%, 0.04%, 0.05%, 0.06%, 0.07%, 0.08%, 0.09%, 0.1%, or as needed to adjust the formulation to the desired pH.

Further and preferably, the pH of the composition of the invention can be adjusted or stabilized with a buffer. Suitable buffers include citrate/citric acid buffers, acetate/acetic acid buffers, phosphate/phosphoric acid buffers, formate/formic acid buffers, propionate/propionic acid buffers, lactate/lactic acid buffers, carbonate/carbonic acid buffers, ammonium/ammonia buffers, and the like. In certain instances, the buffer is an acidic buffer system such as for example, benzocaine. In more preferred instances, the acidic acid buffer system is citric acid or a citric acid salt.

In certain preferred aspects, the buffer system comprises panthenol, either alone or in combination with 3-aminopropanol.

In certain preferred instances, the buffer is present at a concentration of about 0.000001 M, 0.00001 M, 0.0001 M, 0.001 M, 0.0012 M, 0.01 M, 0.012 M, 0.1 M, or 1.0 M. Preferably, this amount is about 0.0010 M, 0.0015 M, 0.002 M, 0.003 M, 0.004 M, 0.005 M, 0.006 M, 0.007 M, 0.008 M, 0.009 M, 0.01 M, 0.012 M, or 0.02 M. More preferably, this amount is about 0.001 M, 0.002 M, 0.003 M, 0.004 M, 0.005 M, 0.006 M, 0.007 M, 0.008 M, 0.009 M, 0.010 M, 0.011 M, 0.012 M, 0.015 M, 0.016 M, 0.017 M, 0.018 M, 0.019 M, 0.02 M, 0.025 M, 0.03 M, 0.035 M, 0.04 M, 0.045 M, 0.05 M, 0.055 M, 0.06 M, 0.065 M, 0.07 M, 0.075 M, 0.08 M, 0.085 M, 0.09 M, 0.095 M, or 0.1 M. Still more preferably, this amount is about 0.10 M, 0.11 M, 0.12 M, 0.13 M, 0.14 M, 0.15 M, 0.16 M, 0.17 M, 0.18 M, 0.19 M, 0.20 M, 0.21 M, 0.22 M, 0.23 M, 0.24 M, 0.25 M, 0.26 M, 0.27 M, 0.28 M, 0.29 M, 0.30 M, 0.31 M, 0.32 M, 0.33 M, 0.34 M, 0.35 M, 0.36 M, 0.37 M, 0.38 M, 0.39 M, 0.40 M, 0.41 M, 0.42 M, 0.43 M, 0.44 M, 0.45 M, 0.46 M, 0.47 M, 0.48 M, 0.49 M, 0.50 M, 0.55 M, 0.60 M, 0.65 M, 0.7 M, 0.75 M, 0.8 M, 0.85 M, 0.9 M, 0.95 M, or 1.0 M. In certain preferred instances, the inventive formulation includes a buffer, and a second pH-adjusting agent (e.g., sodium hydroxide or hydrochloric acid) to adjust the pH of the composition to a desired pH. More preferably, the second pH-adjusting agent comprises two agents (e.g., sodium hydroxide and hydrochloric acid) which are included as needed to adjust the pH of the composition to a desired pH.

The present composition may optionally include one or more of the following: glycerine, at least one antioxidant, one chelating agent, a preservative, a thickening agent, one or more emulsifiers, pharmaceutically acceptable formulation aids, and penetration enhancers. Useful penetration enhancers include, but are not limited to, ethyl alcohol, isopropyl alcohol, or octolyphenylpolyethylene glycol. More preferred penetration enhancers include oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone.

The formulation may be made bacteriostatic for safe application to skin that is compromised by AHZ by the addition of preservatives. For example, a composition can contain 0.001-8%, preferably 0.01-6%, more preferably 0.05-5% by weight of the total composition of a preservative or a combination of preservatives. A variety of preservatives are suitable, including, but not limited to, benzoic acid, benzyl alcohol, benzylhemiformal, benzylparaben, 5-bromo-5-nitro-1,3-diox-ane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, diethylene glycol dimethyl ether (“DEDM”) hydantoin, DEDM hydantoin dilaurate, dehydroacetic acid, dibromopropamidine diisethionate, and 1,3-bis(hydroxymethyl)-5,5-dimethylimidazolidine-2,4-dione (“DMDM”) hydantoin. In certain aspects, the formulations herein may be (i) sterile or essentially free from microorganisms such as bacteria and viruses that can cause infection and (ii) optionally preservative-free.

In certain aspects, the composition of the present invention comprises a preservative, such as propyl paraben or methyl paraben, or combinations thereof.

Preferred antioxidants for use in the present invention may be selected from the group consisting of butylated hydroxytoluene (“BHT”), butylated hydroxyanisole (“BHA”), ascorbyl linoleate, ascorbyl dipalmitate, ascorbyl tocopherol maleate, calcium ascorbate, carotenoids, kojic acid, tocopherol, tocopherol acetate, tocophereth-5, tocophereth-12, tocophereth-18, tocophereth-80, and mixtures thereof.

Preferred chelating agents may be selected from the group consisting of ethylenediamine tetraacetic acid (“EDTA”), diammonium EDTA, dipotassium EDTA, calcium disodium EDTA, hydroxyethylethylenediaminetriacetic acid (“HEDTA”), ethylenediaminetetraacetic acid, mono(triethanolamine) salt (“TEA-EDTA”), tetrasodium EDTA, tripotassium EDTA, trisodium phosphate, diammonium citrate, galactaric acid, galacturonic acid, gluconic acid, glucuronic acid, humic acid, cyclodextrin, potassium citrate, the potassium salt of ethylenediamine-tetra(methylene phosphonic acid) (“EDTMP”), sodium citrate, sodium EDTMP, and mixtures thereof.

In certain instances, one factor that determines the spray-pumpability of the formulation is viscosity. Viscosity is also a factor that determines how well the formulation sticks to the skin or does not run off the skin when applied. In a specific example, the viscosity of the formulation is less than 1000 centipoise at 20° C. In another example, the viscosity of the formulation is less than 500 centipoise at 20° C. In a further example, the viscosity of the formulation is less than 200 centipoise at 20° C. In still an additional example, the viscosity of the formulation is less than 100 centipoise at 20° C. The viscosity of the formulation can be optimized using one or more pharmaceutically acceptable thickening agents that do not significantly interact with the components of the formulation, do not significantly reduce flux of the formulation, and do not cause stinging or irritation. In one example, one or more of the following thickening agents is used: polyacrylic acid polymers, carbomers, cellulose derivatives, poloxamers, poloxamines, dextrans, pectins, natural gums. In one embodiment, cellulose, hydroxyethyl cellulose (“HEC”), hydroxypropyl methyl cellulose (“HMPC”), carboxymethyl cellulose or mixtures thereof are used as a thickening agent.

One preferred embodiment, the present invention provides a composition comprising, consisting essentially of, or consisting of:
an anesthetic active ingredient that is lidocaine hydrochloride;
a citric acid ester that is triethyl citrate;
a non-ionic surfactant that is a member selected from the group consisting of polyoxyethylene (20) sorbitan monolaurate, polyoxyethylene (20) sorbitan monooleate, and a combination thereof;
a polar solvent that is a member selected from the group consisting of diethylene glycol monoethyl ether, panthenol and a combination thereof; and
water.

In one preferred embodiment of the present invention, the anesthetic active ingredient is lidocaine hydrochloride; the non-ionic surfactant is polyoxyethylene (20) sorbitan monolaurate; and the polar solvent is a combination of diethylene glycol monoethyl ether and panthenol. Preferably, the composition further comprises a pH-adjusting agent or buffer.

In yet another preferred embodiment, the present invention provides a composition comprising, consisting essentially of, or consisting of:
an anesthetic active ingredient of lidocaine or lidocaine hydrochloride (e.g. monohydrate) that is present at about 10%-15%, preferably about 10%;

triethyl citrate that is present at about 1%-5%, preferably about 2%; water that is present at about 60%-75%, preferably about 68.2% or 71.2%; panthenol or D-panthenol that is optionally present at about 1%-5%, preferably about 3%;
diethylene glycol monoethyl ether (Transcutol®) that is present at about 5%-15%, preferably about 8.5%;
polyoxyethylene (20) sorbitan monolaurate that is present at about 5%-15%, preferably about 8.1%; and
methyl paraben or propyl paraben that is present at about 0.01%-0.5%, preferably about 0.1%.

In an alternative embodiment, water replaces panthenol or D-panthenol in the above formulation.

### III. Characteristics of the Formulation

A. Application

In one embodiment, the formulation is spray-pumpable. For instance, the formulation may be spray-pumpable into a stream of ballistic droplets or a mist to cover the area of treatment. Ideally, the size of the individual droplets produced is large enough so that there is no or very low risk that they are deposited into the respiratory tract. In one example, the droplet size is larger than 5 to 30 microns or 1 to 5 microns. The size of the droplets can be adjusted to ensure optimal delivery of the formulation to the area of need and optimal safety. For example, parameters of the formulation, such as viscosity, or parameters of the delivery device, such as nozzle shape and size and flow rate, can be adjusted as required.

In certain instances, the present formulations are spray-on formulations (which may include a propellant) or spray-pumpable formulations, which provide many advantages over currently available patch formulations which as previously explained are unsuitable for the treatment of AHZ. The formulations of present invention are easier to apply, cover a larger surface area, are non-stinging and can be applied without touching the skin surface with other than the formulation itself. The skin surfaces to which the formulations of the current invention can be applied include, but are not limited to, skin of the chest region (thoracic), abdomen, the forehead (trigeminal) or wherever the herpes zoster rash occurs. In addition, the formulations can be applied to other surfaces such as mucosal surfaces, genitals, anus, nail surface, wound surface, rash surface, bed sore surface, and diabetes-induced ulcerous skin surface. In certain instances, the methods of use are those that are set forth in U.S. Provisional Patent Application No. 61/112,123, filed Nov. 11, 2008 and entitled “Formulations for the Treatment of Acute Herpes Zoster Pain,” which application is incorporated herein by reference for all purposes. This application was published on May 14, 2010 as WO 2010/054093.

In another embodiment, the inventive formulation is foamable. Qualities such as foam stability, spreadability (i.e., ease of spreading) and appropriate breakability upon application to the skin are desirable features. These characteristics can be measured by conducting foam formation and foam collapsibility experiments. Foam formation (foam height vs time), for example, is predictive of the generation of a sprayable/spreadable foam. The rate of collapsibility is a property relevant to the appropriate administration of the foam. In a preferred embodiment, the foam is a quick-breaking foam with a high rate of collapsibility so that it can be applied to the skin without rubbing. Preferably, the foam collapses with minimal run-off.

As with the spray-pumpable formulations described above, foamable formulations of the present invention are easier to apply, cover a larger surface area, are non-stinging, and can be applied without touching the skin surface with other than the formulation itself. As well, the foamable compositions of the invention are suitable for use on skin surfaces including, but are not limited to, skin of the chest region (thoracic), abdomen, the forehead (trigeminal) or wherever the herpes zoster rash occurs. The formulations can additionally be applied to other surfaces as previously described.

In some aspects, the foamable compositions of the present invention are dispensed from a reservoir using a release assembly (e.g., a hand pump) to dispense an amount of the composition whenever the release assembly is put into action. The amount of the composition dispensed by the pump may or may not be metered to dispense a consistent amount of formulation.

Non-limiting examples of pumps useful in dispensing foamable compositions of the invention include the Rexam M3 foaming head, the Meadwestvaco Ocean T and Ocean H spray heads and any suitable hand soap dispenser. However, the compositions of the invention are not limited to being dispensed from only one type of dispenser or through only one type of hand pump. Further, the dispenser or pump head may include additional or altered features that assist in optimizing foam stability. These features include, but are not limited to, the inclusion of meshes in the pump head and varied dip tube and nozzle lengths.

B. Non-Stinging/Non-Irritating Features

Advantageously, the formulations of the present invention are non-stinging and/or non-irritating to the subject. After application to the skin, any skin reaction is imperceptible or sufficiently mild as to not preclude topical or transdermal administration. In other words, the perception of stinging, pain, or of a distinct discomfort to the user when applied is imperceptible or de minimus. A stinging assay can be used to assess whether the novel topical formulations described herein produce a sensory perception of stinging. In a preferred embodiment, the stinging or pain sensation of the inventive formulations applied to the skin is imperceptible.

Likewise, an irritancy study can be conducted to assess whether the novel topical formulations described herein cause irritation of the skin. In a preferred embodiment, the formulation is classified as a low-irritancy topical formulation when, following its application to the skin, there is an absence of an acute irritation response (erythema/edema) after 72 hours.

C. Stability

In certain aspects of the instant invention, the topical formulations have the advantage of maintaining chemical and/or physical stability over time, even where the concentration of the active has been increased or there is a tendency for components (e.g., triethyl citrate) to degrade. In Examples 18, 20, and 21, for instance, the chemical and physical attributes of certain preferred topical formulations were monitored over the course of a three-month period. Surprisingly, the inclusion of panthenol (sometimes in combination with a buffer, e.g., citric acid and its salts, or a pH-adjusting agent, e.g., 3-aminopropanol) provided formulations that were unexpectedly stable. Without being bound by theory, panthenol's ability to reduce the rate of degradation of triethyl citrate may be attributed to the production of aminopropanol via hydrolysis of panthenol. This functionality results in a buffering effect which allows panthenol to stabilize the pH of the formulation and consequently reduce the rate of hydrolysis of triethyl citrate.

In certain aspects of the invention, the pharmaceutical composition is substantially stable with respect to its chemical and/or physical attributes over a predetermined period of time. The measurable attributes may include, but are not limited to, pH, percentage of active, percentage of impurities, or visual attributes such as color and the presence of particulates. In other aspects the invention, the pharmaceutical composition is substantially stable following storage for about 4, 8 or 12 weeks at 25° C. In still other aspects of the invention, the pharmaceutical composition is substantially stable following storage for about 4, 8 or 12 weeks at 40° C. In still other aspects of the invention, the pharmaceutical composition is substantially stable following storage for about 4, 8 or 12 weeks at 70° C.

### IV. Methods of Use

In certain aspects, the compositions and formulations of the invention are particularly suited for use in treating pain associated or resulting from an acute herpes zoster infection. In certain preferred aspects, the methods employ an anesthetic active agent in an effective amount to achieve analgesia without or with minimal anesthesia. The formulation is applied to the site of pain typically once, twice, three or four times or as needed per day.

Various modes of application of the inventive formulations can be employed to ensure that a level of an analgesic active agent is maintained for a time sufficient to substantially reduce the pain accompanying AHZ during the application and frequently after the application has been terminated. The pain accompanying AHZ can be throbbing, stabbing, burning, or lancinating in character, is commonly associated with allodynia, and has been shown to be moderate to severe in intensity within 72 hours of rash onset.

In other aspects, the compositions and formulations of the invention are particularly suited for use in treating pain associated with postherpetic neuralgia (“PHN”). The invention provides a method for administering a local anesthetic agent to a patient to treat or prevent pain. The method involves topically administering a pharmaceutical composition as described herein to treat patients suffering from pain associated with a skin condition or disorder, e.g., an insect bite, muscle pain, arthritis, fibromyalgia, myofascial pain, allergic reaction, rash (e.g., a rash caused by poison oak or poison ivy), itch, blister, sore nail, corn, mechanical puncture (e.g., catheterization and needle injection), laser treatment, or any combination thereof.

The method may also be used to treat patients suffering from breakthrough pain, migraine, neuropathic pain, and various other types of intense pain. In addition, the compositions and systems of the invention may be administered with a wound dressing to treat burns, wounds and scrapes.

Advantageously, the compositions and drug delivery systems described herein can also be used as part of a pre-treatment regimen used to prevent or minimize the pain associated with other topical therapies, medical procedures or cosmetic procedures.

### V. Examples

### Methods:

Franz diffusion cell (“FDC”) experiments were used to analyze lidocaine flux rates from varying formulations across a substrate membrane. Franz diffusion cells are a common and well known method for measuring transdermal flux rates. The general Franz cell procedure is described in Franz, T. J., Percutaneous absorption: on the relevance of in vitro data: J. Invest Dean, 64:190-195 (1975). The following was the methodology used in the present Examples.

Franz cells with a 3 ml receptor well volume were used in conjunction with split thickness cadaver skin (0.015″-0.018″, AlloSource of Centennial, Colo.) or dermatomed porcine skin (Lampire Biological Laboratories, of Pipersville, Pa.). The donor wells of the Franz cells had an area of ˜0.5 cm². Receptor wells were filled with isotonic phosphate buffered saline (PBS) doped with 0.01% sodium azide. The flanges of the Franz cells were coated with vacuum grease to ensure a complete seal and were clamped together with uniform pressure using a pinch clamp (SS #18 VWR 80073-350). After the Franz cells were assembled, the skin was allowed to pre-hydrate for ˜45 minutes. The quantity of formulation applied to the substrate varied from 2 mg/cm² (considered finite dose) to 200 mg/cm² (considered infinite dose). The Franz cells were maintained at 32° C. by placement in a humidified incubator. The receptor wells of the Franz cells were agitated at all times with a stir bar. Sample aliquots were drawn from the receptor wells at varying time points and replaced with fresh buffer. Measurements for each formulation were carried out in six-fold replicates. The concentrations of the active in the sample aliquots were analyzed using high performance liquid chromatography (“HPLC”). In certain experiments, Lidoderm® patch was used as a control. A Lidoderm® patch is comprised of an adhesive material containing 5% lidocaine base, which is applied to a non-woven polyester felt backing and covered with a polyethylene terephthalate (“PET”) film release liner. The release liner is removed prior to application to the skin. The size of the patch is 10 cm×14 cm, which can be cut for example, into a circle with a diameter equal to the donor well diameter. Each adhesive patch contains 700 mg of lidocaine base (50 mg per gram adhesive) in an aqueous base (note that as many of the example embodiments of the present invention are prepared using lidocaine hydrochloride, rather than lidocaine base, in the example tables provided following, the weight percentage of lidocaine base in Lidoderm® is not explicitly listed; it is 5% in all cases). Lidoderm® also contains the following inactive ingredients: dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacrylate, D-sorbitol, tartaric acid, and urea. For experiments wherein the retention of lidocaine was measured in the skin, the skin was collected, washed of excess formulation on the stratum corneum, then homogenized in a ethanol solution. Over the period of one day, the lidocaine was extracted from the skin into the ethanol solution. An aliquot of the ethanol was then taken and measured for lidocaine concentration.

Typical formulation compositions and permeation behaviors are given in the following tables and figures.

### Example 1

The following example illustrates the use of ethyl acetate in a formulation with lidocaine hydrochloride.

TABLE 1

| | Formulation name | | | | | | |
|---|---|---|---|---|---|---|---|
| | Lidoderm ® | EA12-Cl | EA22-Cl | EA26-Cl | EA31-Cl | EA34-Cl | EA37-Cl |
| | Dosing (μl) | | | | | | |
| | | 3.0 | 3.0 | 3.0 | 3.0 | 3.0 | 3.0 |
| Percentages in | w/w | w/w | w/w | w/w | w/w | w/w | w/w |
| Propylene Glycol | | 10 | | | 10 | | 10 |
| Water | | 76 | 76 | 76 | 76 | 76 | 76 |
| Lidocaine HCl | | 10 | 10 | 10 | 10 | 10 | 10 |
| monohydrate | | | | | | | |
| Ethyl acetate | | 5 | 5 | 5 | 5 | 5 | 5 |
| Transcutol | | | 10 | 10 | | | |
| Tween 20 | | | | 9 | | | 9 |
| Tween 80 | | 9 | 9 | | 9 | 9 | |
| Glycerine | | | | | | 10 | |

TABLE 2
Accumulated Doses (μg/cm²)

| Time | Lidoderm ® | EA12-Cl | EA22-Cl | EA26-Cl | EA31-Cl | EA34-Cl | EA37-Cl |
|---|---|---|---|---|---|---|---|
| 2 hrs | 12.4 | 10.6 | 30.4 | 40.2 | 26.8 | 42.4 | 62.1 |
| 3 hrs | 20.5 | 53.9 | 46.0 | 65.1 | 50.4 | 37.0 | 50.8 |
| 20 hrs | 365.4 | 424.9 | 385.4 | 366.6 | 261.5 | 196.8 | 403.4 |
| 24 hrs | 207.9 | 415.6 | 261.6 | 175.9 | 126.1 | 118.8 | 312.0 |

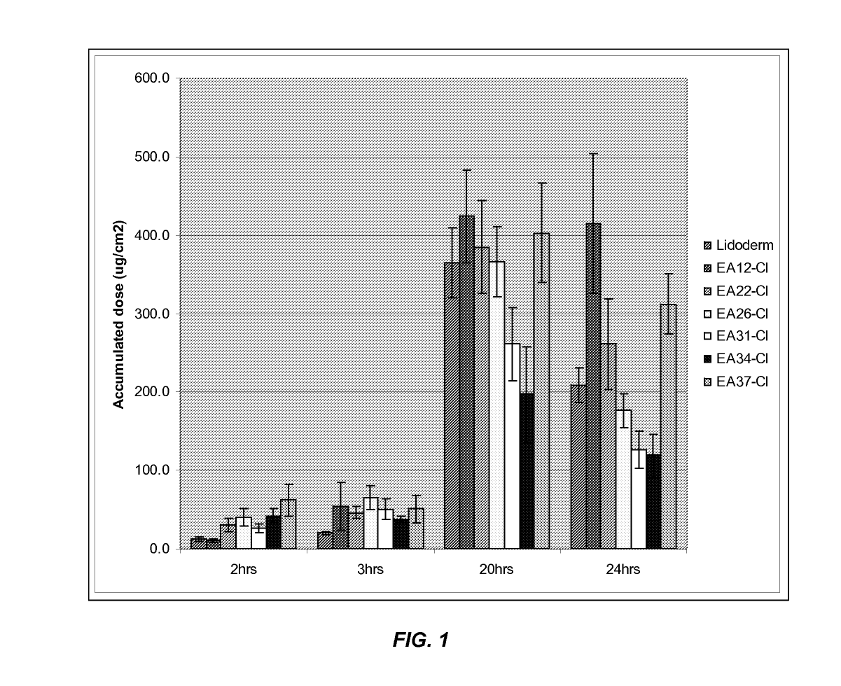

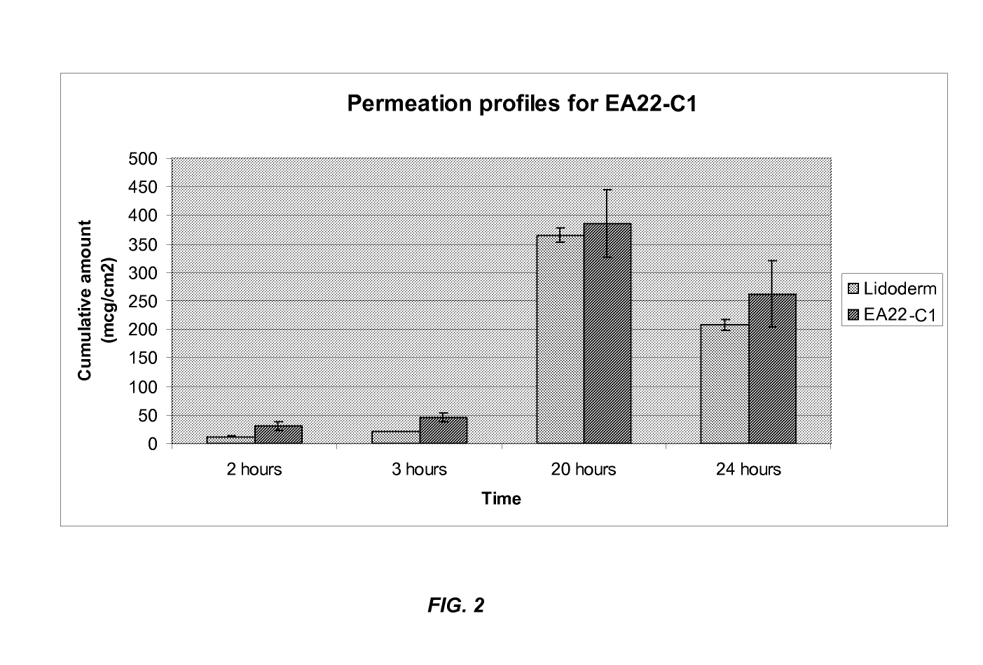

The results of the penetration study are shown in Table 2 and FIG. 1. A typical permeation profile for EA 22 is given in FIG. 2 and Table 2. It is apparent from the FIG. 2 that the cumulative lidocaine flux from formulation EA 22 at each time point is similar to that from Lidoderm®.

### Example 2

The following example illustrates the use of ethyl acetate in a formulation with lidocaine hydrochloride.

TABLE 3

| | Formulation name | | | | | | |
|---|---|---|---|---|---|---|---|
| | Lidoderm ® | EA12-Cl | EA12-Cl fr | EA52-Cl | EA55-Cl | EA58-Cl | EA61-Cl |
| | Dosing (μl) | | | | | | |
| | | 3.0 | 3.0 | 3.0 | 3.0 | 3.0 | 3.0 |
| Percentages in | w/w | w/w | w/w | w/w | w/w | w/w | w/w |
| Propylene glycol | | 10 | 10 | | | 10 | |
| Water | | 76 | 76 | 76 | 76 | 76 | 76 |
| Lidocaine HCl | | 10 | 10 | 10 | 10 | 10 | 10 |
| monohydrate | | | | | | | |
| Ethyl acetate | | 5 | 5 | 5 | 5 | 5 | 5 |
| Transcutol | | | | 10 | | | |
| Tween 80 | | 9 | 9 | | | | |
| Isopropyl alcohol | | | | | 10 | | |
| Glycerine | | | | | | | 10 |
| Tween 60 | | | | 9 | 9 | 9 | 9 |

fr = freshly prepared

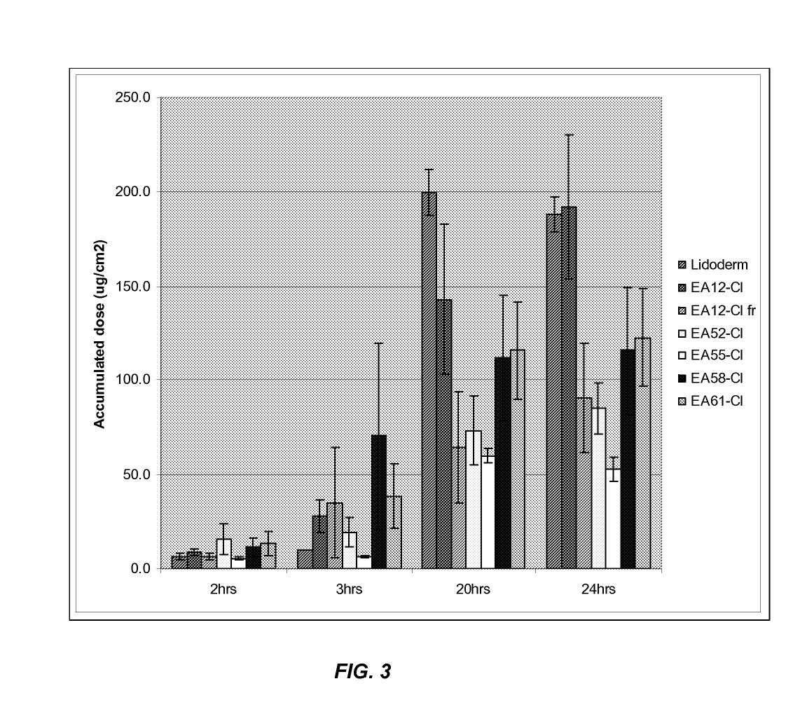

The permeation results show that the delivery of lidocaine through the skin from the inventive formulations are similar to that from Lidoderm®. Polyols such as glycerine reduce permeation. The permeation profiles results are shown in FIG. 3. Incorporation of nonionic surfactants results in different permeation behaviors. For example, Tween 60 reduces permeation.

### Example 3

This Example illustrates the use of ethyl acetate in combination with lecithin in formulations with lidocaine hydrochloride.

TABLE 4

| | Formulation name | | | | | | |
|---|---|---|---|---|---|---|---|
| | Lidoderm ® | EA12 | EA43-egg | EA43-soy | EA-46egg | EA-49egg | EA49-soy |
| | Dosing (μl) | | | | | | |
| | | 3.0 | 3.0 | 3.0 | 3.0 | 3.0 | 3.0 |
| Percentages in | w/w | w/w | w/w | w/w | w/w | w/w | w/w |
| Propylene glycol | | 10 | | | | | |
| Water | | 76 | 75 | 75 | 75 | 75 | 75 |
| Lidocaine HCl | | 10 | 10 | 10 | 10 | 10 | 10 |
| monohydrate | | | | | | | |
| Ethyl acetate | | 5 | 5 | 5 | 5 | 5 | 5 |
| Transcutol | | | 10 | 10 | | 10 | 10 |
| Tween 20 | | | | | 9 | 9 | 9 |
| Tween 80 | | 9 | 9 | 9 | | | |
| Isopropyl alcohol | | | | | 10 | | |
| Lecithin egg | | | 1 | | 1 | 1 | |
| Lecithin soy | | | | 1 | | | 1 |

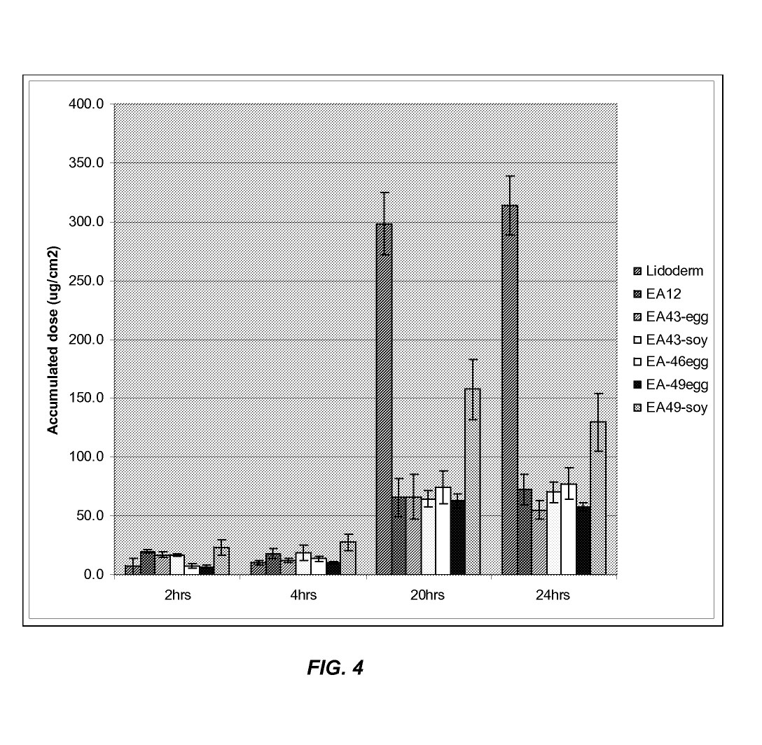

To modulate permeation, soy lecithin was added to the formulation. The permeation profiles results are shown in FIG. 4.

### Example 4

The following example illustrates the use of polysorbates or other components in formulations with lidocaine.

TABLE 5

| | Formulation name | | | | | | | | | |
|---|---|---|---|---|---|---|---|---|---|---|
| | Lidoderm ® | EA87 | EA89 | EA90 | EA93 | EA95 | EA96 | TC1 | TC2 | TC3 |
| | Dosing (μl) | | | | | | | | | |
| | patch | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % |
| Tween 80 | | 8.1 | 8.1 | 8.1 | 8.1 | 8.1 | 8.1 | 8.1 | 8.1 | 8.1 |
| Transcutol | | 0 | 9 | 9 | 9 | 9 | 9 | 9 | | 9 |
| Isopropyl | | | | | | | | | 9 | |
| alcohol | | | | | | | | | | |
| Propylene | | | 9 | 9 | | 9 | | | 9 | |
| glycol | | | | | | | | | | |
| Glycerine | | 9 | | | 9 | | | 9 | | 9 |
| Water | | | | | | | | 54.9 | 54.9 | 54.9 |
| 5% Urea | | 54.9 | 54.9 | 54.9 | | | | | | |
| (aqueous | | | | | | | | | | |
| solution) | | | | | | | | | | |
| 5% Choline | | | | | 54.9 | 54.9 | 68.4 | | | |
| chloride | | | | | | | | | | |
| (aqueous | | | | | | | | | | |
| solution) | | | | | | | | | | |
| Lidocaine | 5 | 10 | 10 | 10 | 10 | 10 | 10 | 10 | 10 | 10 |
| Triethyl citrate | | | | | | | | 4.5 | 4.5 | 4.5 |
| PEG-Castor oil | | 4.5 | 4.5 | 4.5 | 4.5 | 4.5 | | 4.5 | 4.5 | 4.5 |
| Ethyl acetate | | 4.5 | 4.5 | 4.5 | 4.5 | 4.5 | 4.5 | | | |

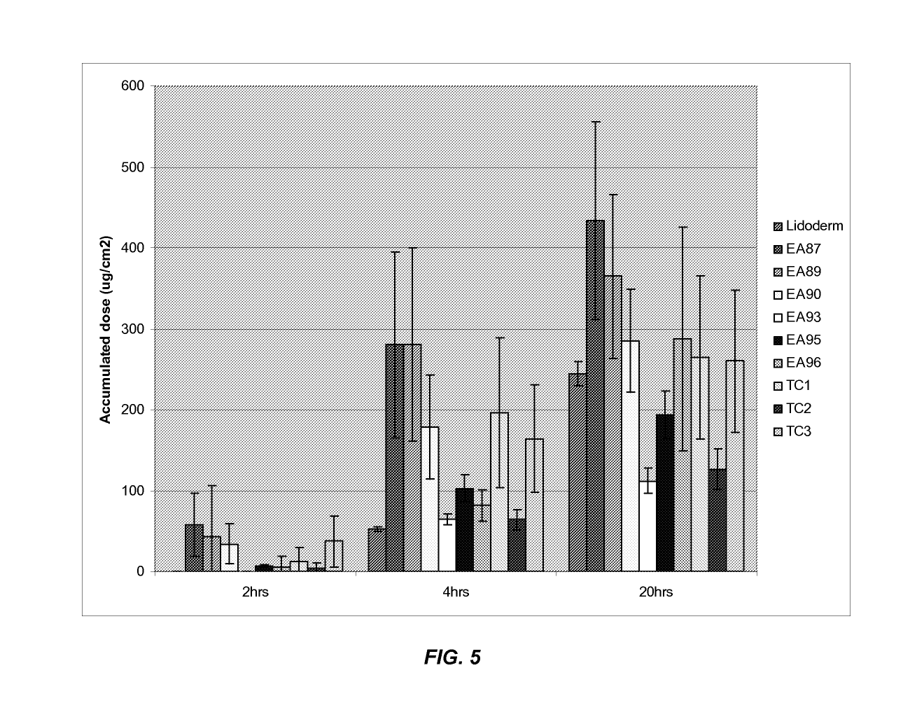

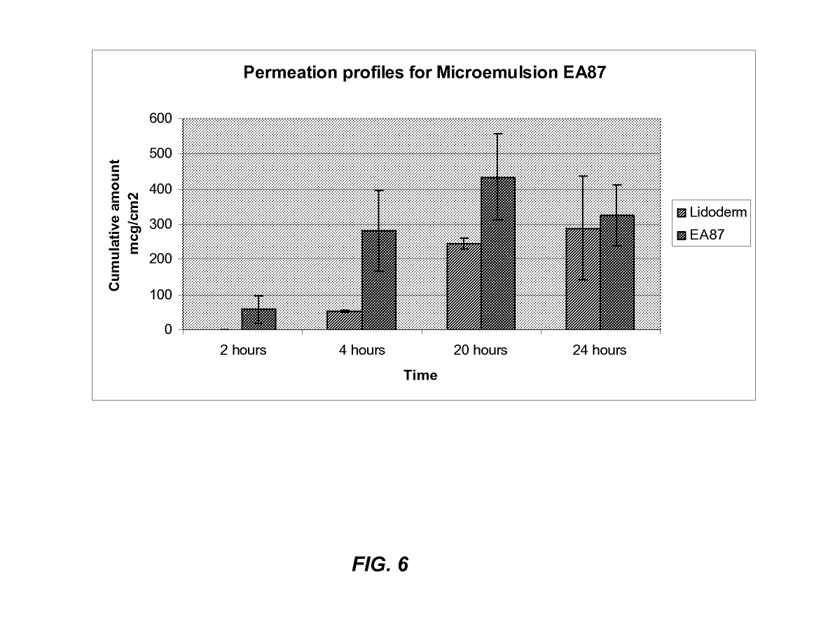

In addition, the incorporation of some keratolytic agents such as urea or choline chloride was evaluated. As in Example 3 with ethyl acetate, formulations with a surfactant based chassis containing co-surfactant, together with permeation enhancing agents such as urea and choline chloride were examined. The permeation profiles results are shown in FIG. 5. The permeation profile of EA 87 compared to Lidoderm® is shown in FIG. 6.

### Example 5

The following examples illustrate the use of triethyl citrate with isotonic sucrose in formulations with lidocaine hydrochloride, to enhance permeation.

TABLE 6

| | Formulation name | | | | | | | | | | | |
|---|---|---|---|---|---|---|---|---|---|---|---|---|
| | Lidoderm ® | TC4 | TC5 | TC6 | TC7 | TC8 | TC9 | TC10 | TC11 | TC12 | EA88 | EA94 |
| | Dosing (μl) | | | | | | | | | | | |
| | patch | 3.0 | 3.0 | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % | wt/wt % | wt/wt % |
| Triethyl citrate | | 4.5 | 4.5 | 4.5 | 4.5 | 4.5 | 4.5 | 4.5 | 4.5 | 4.5 | | |
| PEG-Castor oil | | | | | 4.5 | 4.5 | 4.5 | | | | 4.5 | 4.5 |
| Tween 80 | | 8.1 | | | 8.1 | 8.1 | 8.1 | 8.1 | | | 8.1 | 8.1 |
| Tween 20 | | | 8.1 | 8.1 | | | | | 8.1 | 8.1 | | |
| Transcutol | | 9 | | 9 | 9 | | 9 | 9 | | 9 | | |
| Isopropyl | | | 9 | | | 9 | | | 9 | | 9 | 9 |
| alcohol | | | | | | | | | | | | |
| Propylene | | | | | | 9 | 9 | | | | 9 | 9 |
| Glycol | | | | | | | | | | | | |
| Glycerine | | | | | 9 | | | | | | | |
| Water | | 68.4 | 68.4 | 68.4 | | | | | | | | |
| Lidocaine HCl | | 10 | 10 | 10 | 10 | 10 | 10 | 10 | 10 | 10 | 10 | 10 |
| monohydrate | | | | | | | | | | | | |
| 5% Sucrose | | | | | 54.9 | 54.9 | 54.9 | 68.4 | 68.4 | 68.4 | | |
| (aqueous | | | | | | | | | | | | |
| solution) | | | | | | | | | | | | |
| Ethyl acetate | | | | | | | | | | | 4.5 | 4.5 |
| 5% Urea | | | | | | | | | | | 54.9 | |
| 5% Choline | | | | | | | | | | | | 54.9 |
| chloride | | | | | | | | | | | | |

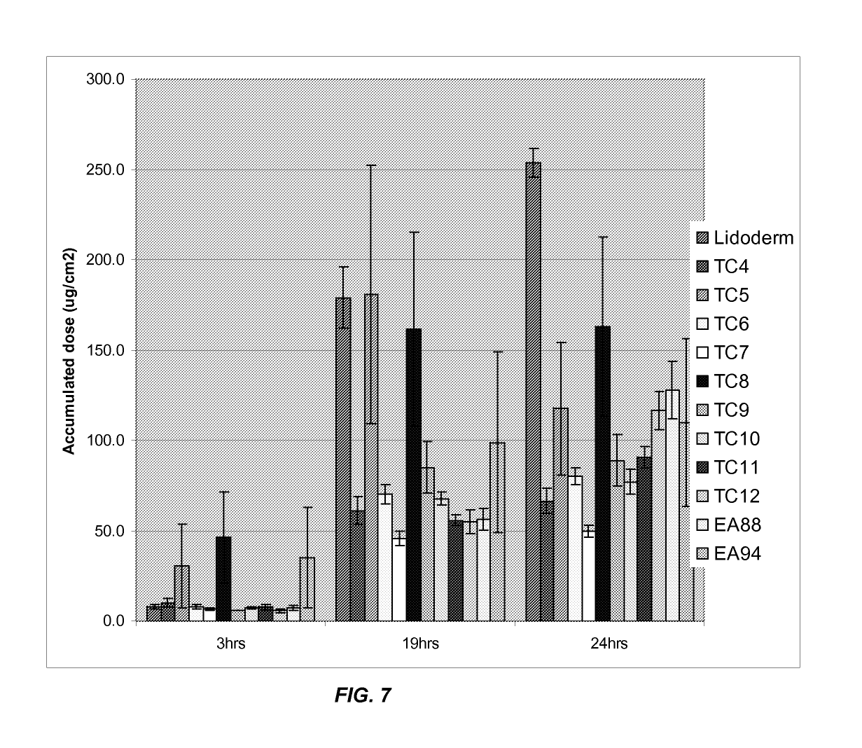

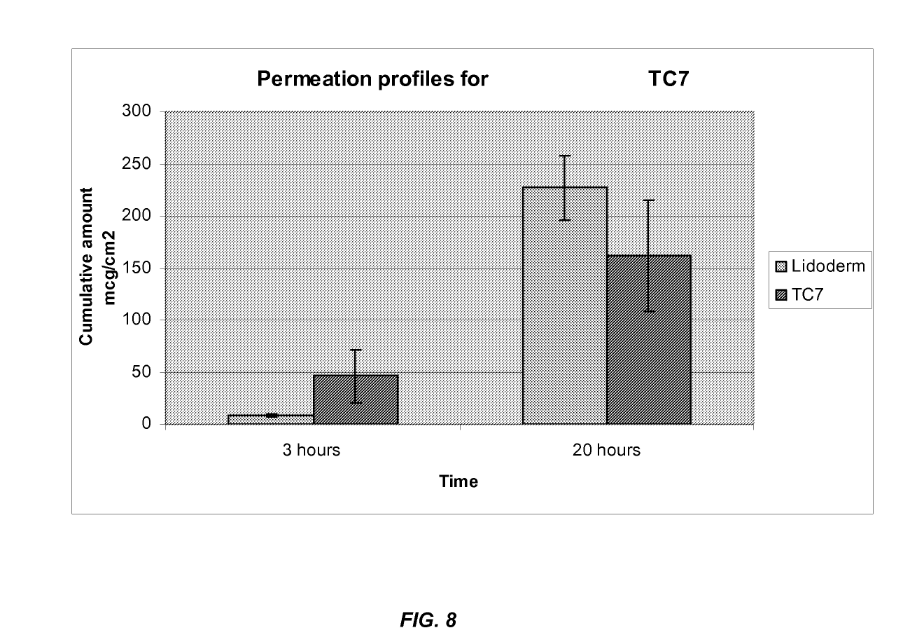

The permeation profiles are shown in FIG. 7. An example of individual permeation of this series formulation is shown in FIG. 8.

### Example 6

The following example illustrates the use of triethyl citrate in formulations with lidocaine hydrochloride and other components.

TABLE 7

| | Formulation name | | | | | | | | | | | |
|---|---|---|---|---|---|---|---|---|---|---|---|---|
| | Lidoderm | TC19 | TC20 | TC21 | TC22 | TC23 | TC24 | TC25 | TC26 | TC27 | TC28 | TC29 |
| | Dosing (μl) | | | | | | | | | | | |
| | patch | 3.0 | 3.0 | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % | wt/wt % | wt/wt % |
| Triethyl citrate | | 4.5 | 4.5 | 4.5 | 4.5 | 4.5 | 4.5 | 4.5 | 4.5 | 4.5 | 4.5 | 4.5 |
| Tween 80 | | 8.1 | 8.1 | 8.1 | 8.1 | 8.1 | 8.1 | 8.1 | 8.1 | 8.1 | 8.1 | 8.1 |
| Transcutol | | | 5 | 6 | 7 | | 5 | | 5 | 5 | | |
| Propylene glycol | | 9 | 4 | 3 | 2 | | | | | | 2 | |
| PEG300 | | | | | | 9 | 4 | | | | | |
| Glycerine | | | | | | | | 9 | 4 | 2 | | |
| Butanediol | | | | | | | | | | | 9 | |
| Hexanediol | | | | | | | | | | | | 9 |
| Water | | 68.4 | 68.4 | 68.4 | 68.4 | 68.4 | 68.4 | 68.4 | 68.4 | 68.4 | 68.4 | 68.4 |
| Lidocaine HCl | | 10 | 10 | 10 | 10 | 10 | 10 | 10 | 10 | 10 | 10 | 10 |
| monohydrate | | | | | | | | | | | | |

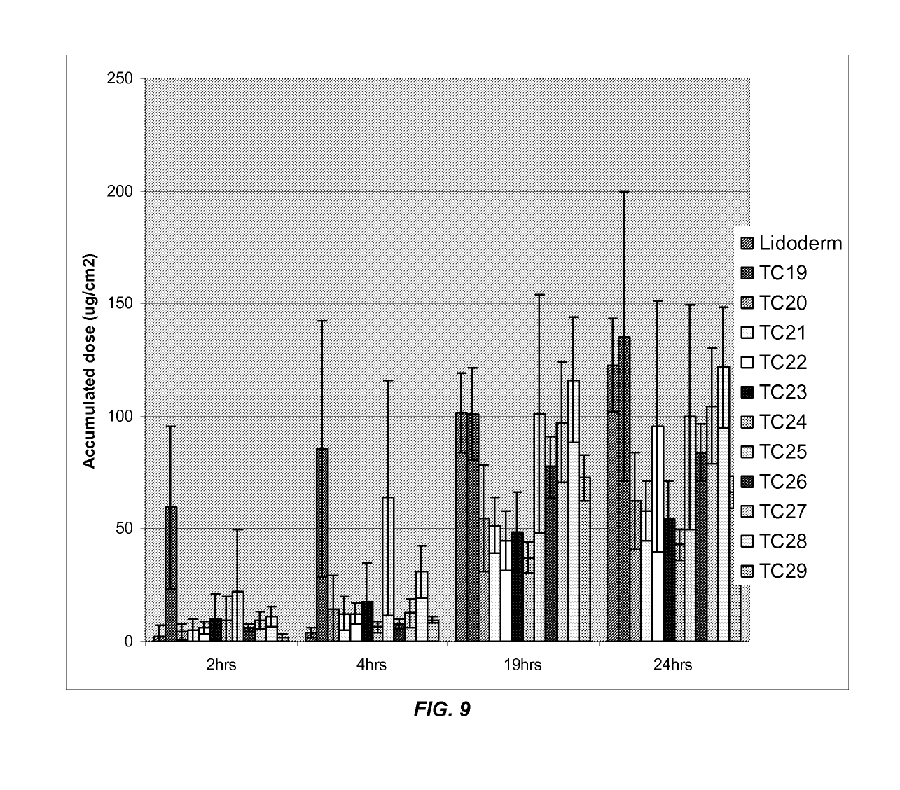

Several variations were examined in terms of their ability to modulate percutaneous lidocaine flux. The permeation profiles results are shown in FIG. 9. Notably, TC 28 shows similar behavior to TC 19.

### Example 7

The following examples illustrate the use of triethyl citrate in formulations with lidocaine hydrochloride and D-panthenol.

TABLE 8

| | Formulation name | | | | | | | | | | | |
|---|---|---|---|---|---|---|---|---|---|---|---|---|
| | Lidoderm ® | TC34 | TC35 | TC36 | TC37 | TC38 | TC39 | TC40 | TC41 | TC42 | TC43 | TC44 |
| | Dosing (μl) | | | | | | | | | | | |
| | | 3.0 | 3.0 | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % | wt/wt % | wt/wt % |
| Triethyl citrate | | 4.5 | 4.5 | 4.5 | 4.5 | 4.5 | 4.5 | 4.5 | 4.5 | 4.5 | 4.5 | 4.5 |
| Tween 80 | | 8.1 | 8.1 | 8.1 | 8.1 | 8.1 | | | | | | |
| Tween 20 | | | | | | | 8.1 | 8.1 | 8.1 | 8.1 | 8.1 | 8.1 |
| Transcutol | | | 5 | 6 | 7 | 5 | | 5 | | 7 | 5 | 5 |
| D-Panthenol | | 9 | 4 | 3 | 2 | 2 | 9 | 4 | 9 | 2 | 2 | 2 |
| Glycerine | | | | | | 2 | | | | | 2 | |
| Propylene | | | | | | | | | | | | 2 |
| Glycol | | | | | | | | | | | | |
| Water | | 68.4 | 68.4 | 68.4 | 68.4 | 68.4 | 68.4 | 68.4 | 68.3 | 68.4 | 68.4 | 68.4 |
| Propyl paraben | | | | | | | | | 0.1 | | | |
| Lidocaine HCl | | 10 | 10 | 10 | 10 | 10 | 10 | 10 | 10 | 10 | 10 | 10 |
| monohydrate | | | | | | | | | | | | |

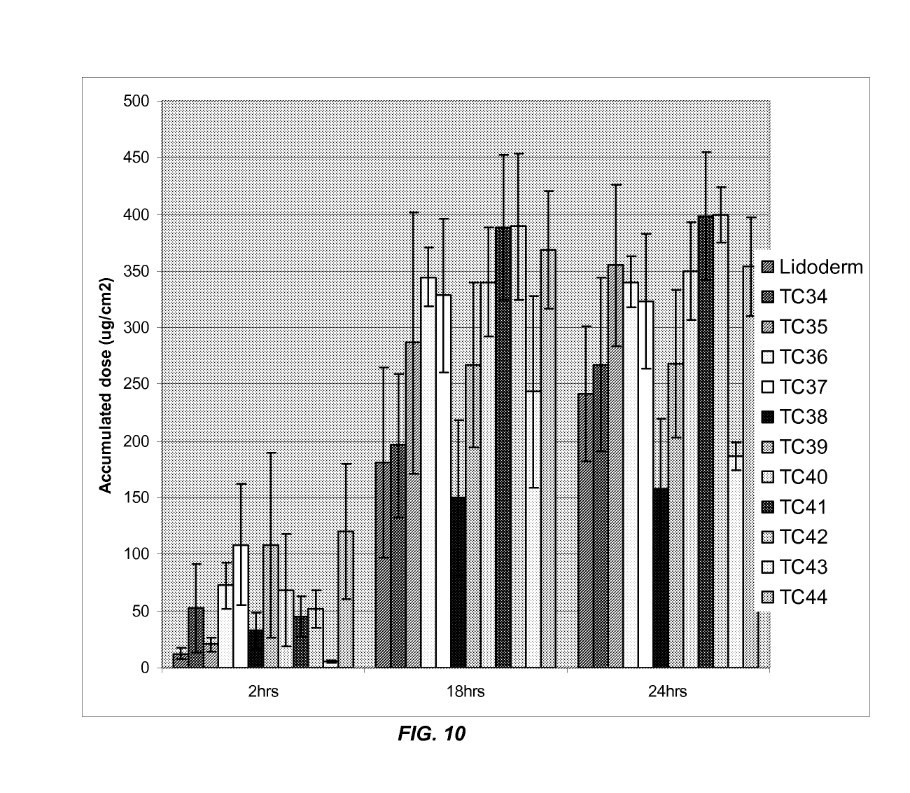

To further enhance the delivery and reduce irritation, D-panthenol USP was used as a humectant, skin protectant, and mild alcohol. The permeation profiles results are shown in FIG. 10. In general, D-panthenol does not decrease the permeation, however, glycerine in combination with D-panthenol does limit permeation.

### Example 8

The following examples illustrate the use lidocaine hydrochloride with various triethyl citrate levels.

TABLE 9

| | Formulation name | | | | | | | | | | |
|---|---|---|---|---|---|---|---|---|---|---|---|
| | Lidoderm | TC41 | TC45 | TC46 | TC49 | TC50 | TC51 | TC52 | TC53-D | TC56 | TC63 |
| | Dosing (μl) | | | | | | | | | | |
| | | 3.0 | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % | wt/wt % |
| Water | | 68.3 | 70.8 | 65.8 | 68.2 | 68.2 | 68.2 | 68.2 | 68.2 | 68.2 | 68.2 |
| D-Panthenol | | 9 | 9 | 9 | 3 | 4 | 3 | 5.5 | 3 | 8.5 | 9 |
| Propyl paraben | | 0.1 | 0.1 | 0.1 | 0.1 | 0.1 | 0.1 | 0.1 | 0.1 | 0.1 | 0.1 |
| Transcutol | | | | | 6 | 6 | 7 | 6 | 8.5 | 3 | |
| Triethyl citrate | | 4.5 | 4.5 | 4.5 | 4.5 | 3.5 | 3.5 | 2 | 2 | 2 | 4.5 |
| Tween 20 | | 8.1 | 8.1 | 8.1 | 8.1 | 8.1 | 8.1 | 8.1 | 8.1 | 8.1 | 8.1 |
| Lidocaine HCl | | 10 | 7.5 | 12.5 | 10 | 10 | 10 | 10 | 10 | 10 | 10 |
| monohydrate | | | | | | | | | | | |
| Methyl paraben | | | | | 0.1 | 0.1 | 0.1 | 0.1 | 0.1 | 0.1 | 0.1 |

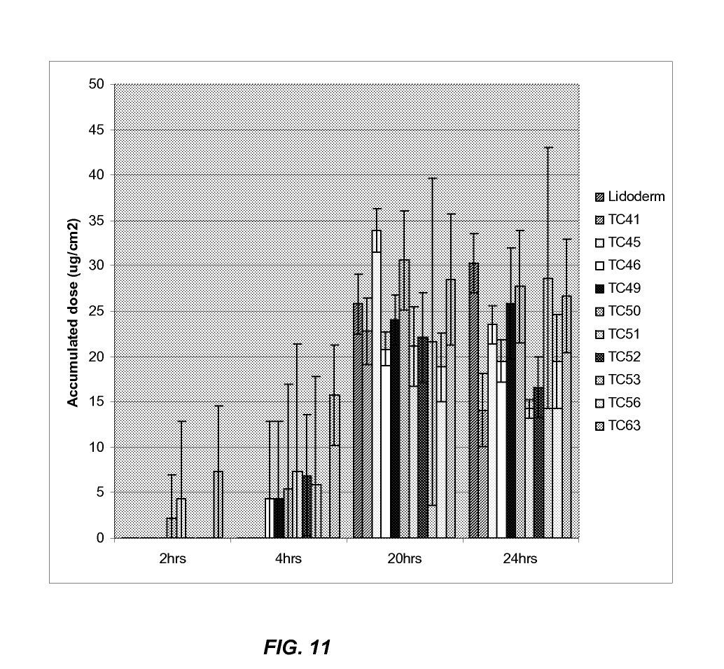

Advantageously, these triethyl citrate formulations are discovered, unexpectedly, to provide transdermal lidocaine fluxes that are comparable to those obtained with Lidoderm®. In addition, due to their lower viscosity, triethyl citrate formulations are readily sprayable. To examine the effect of varying triethyl citrate concentrations on the lidocaine permeation, formulations containing various amounts of triethyl citrate were prepared (Table 9) and assessed. The results are shown in FIG. 11. It is evident that at low levels of triethyl citrate good lidocaine permeation is achieved.

### Example 9

This Example illustrates the use of triethyl citrate in formulations with lidocaine hydrochloride and other components.

TABLE 10

| Formulation name | Lidoderm ® | TC49 | TC71 | Tc72 |
|---|---|---|---|---|
| Dosing (μl) | | 5.0 | 5.0 | 5.0 |
| Percentages in | wt/wt % | wt/wt % | wt/wt % | wt/wt % |
| Water | | 68.2 | 70.7 | 63.2 |
| D-Panthenol | | 3 | 3 | 3 |
| Propyl paraben | | 0.1 | 0.1 | 0.1 |
| Transcutol | | 6 | 6 | 6 |
| Triethyl Citrate | | 4.5 | 4.5 | 4.5 |
| Tween 20 | | 8.1 | 8.1 | 8.1 |
| Lidocaine HCl | | 10 | 7.5 | 15 |
| monohydrate | | | | |
| Methyl paraben | | 0.1 | 0.1 | 0.1 |

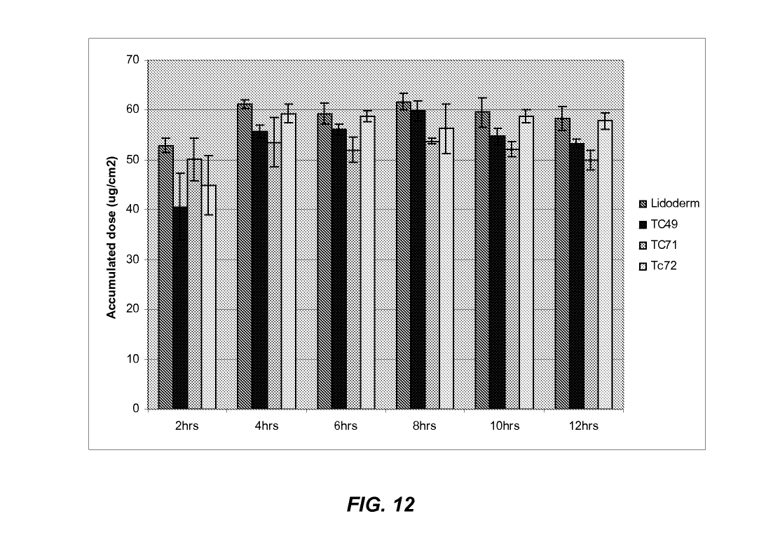

The permeation profile results obtained for the formulations provided in Table 10 are shown in FIG. 12.

### Example 10

This Example illustrates the use of triethyl citrate in formulations with lidocaine hydrochloride.

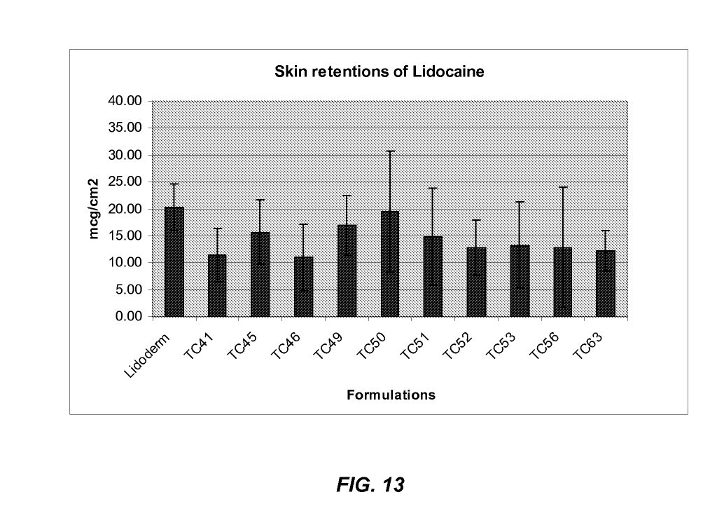

The results provided in Example 8 indicate that the present inventive formulations (TC type) exhibit similar lidocaine permeation behavior to that obtained with Lidoderm® as a control. Of the triethyl citrate based formulations, TC 53 is a preferred formulation. To further assess the effectiveness of TC type formulations, skin retention studies were also performed. All formulations exhibited similar levels of lidocaine retention in the skin (FIG. 13). Dosing was performed using 5 μl.

Further lidocaine permeation measurements were performed based on three formulations provided in Table 9, as per Table 11. The values were calculated using general equations and conditions and the data collected up to 8 hours with 3 μl application.

TABLE 11

| Formulation | Flux (μg/cm2/hr) | Lag time (hr) |
|---|---|---|
| Lidoderm ® | 13.02 | .95 |
| TC 52 | 17.57 | .21 |
| TC 53 | 12.98 | .23 |
| TC 56 | 11.95 | 0 |

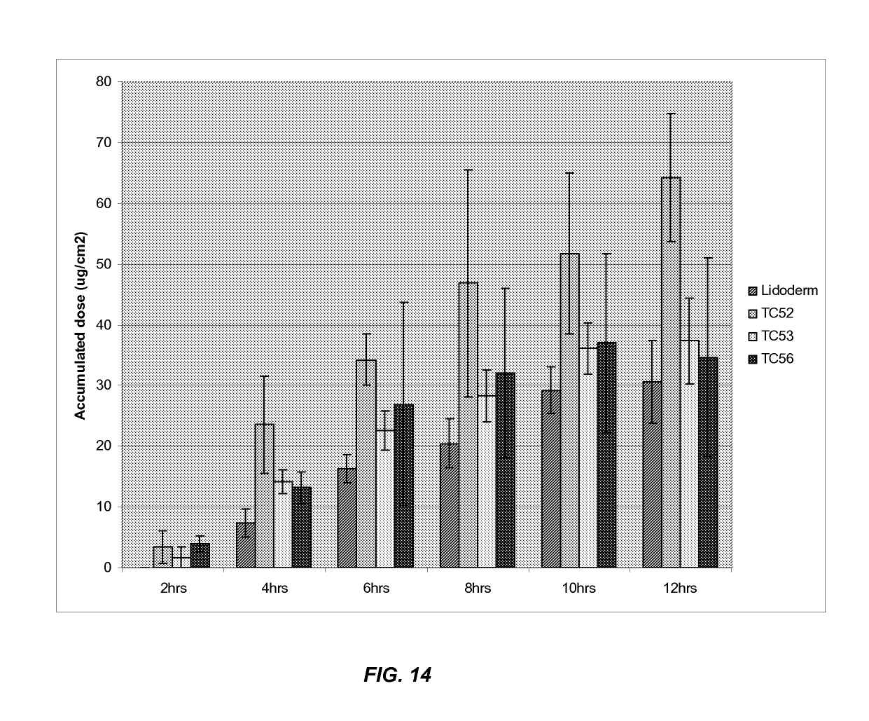

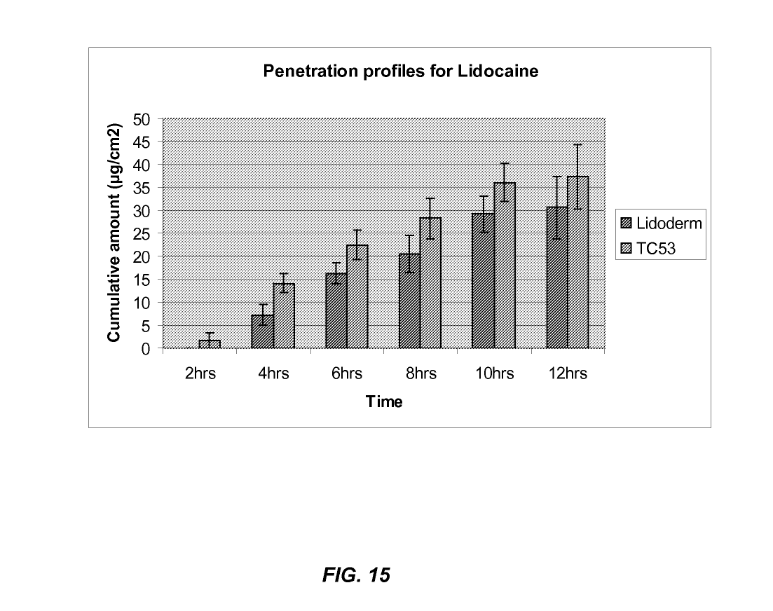

When compared to Lidoderm®, the lidocaine permeation profiles obtained with these TC type formulations are similar (FIG. 14). A comparative time dependent permeation study was also performed using Lidoderm® patch and TC53. At each time point until study completion, the applied formulation (Lidoderm® patch or TC53 sample) was removed and then an extra 3 microliters of formulation, or fresh patch, was added. The results are shown in FIG. 15. The results show that the TC53 inventive formulation provides a lidocaine permeation profile similar to that found with Lidoderm®.

### Example 11

This Example illustrates the use of ethyl acetate in formulations with lidocaine hydrochloride.

TABLE 12

| Formulation name | Lidoderm ® | TC76 | TC79 | TC80 | TC81 |
|---|---|---|---|---|---|
| Dosing (μl) | | 3.0 | 3.0 | 3.0 | 3.0 |
| Percentages in | wt/wt % | wt/wt % | wt/wt % | wt/wt % | wt/wt % |
| Water | | 68.2 | 68.2 | 68.2 | 68.2 |
| D-Panthenol | | 5.8 | 11.6 | | 5.8 |
| Propyl paraben | | 0.1 | 0.1 | 0.1 | 0.1 |
| Transcutol | | 5.8 | | 11.6 | 5.8 |
| Tween 20 | | | 8 | 8 | 8 |
| Tween 80 | | 8 | | | |
| Lidocaine HCl | | 10 | 10 | 10 | 10 |
| monohydrate | | | | | |
| Methyl paraben | | 0.1 | 0.1 | 0.1 | 0.1 |
| Ethyl Acetate | | 2 | 2 | 2 | 2 |

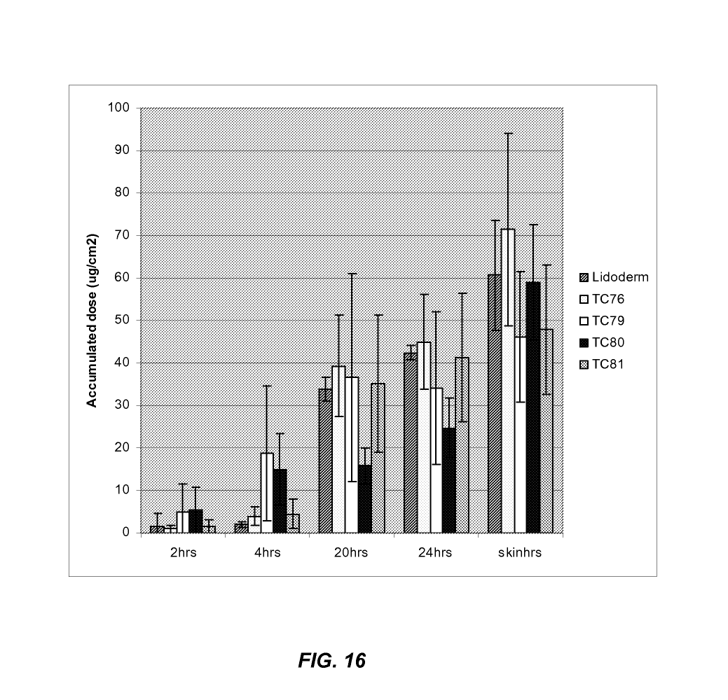

The results are shown in FIG. 16.

### Example 12

This Example further illustrates the use of ethyl acetate in formulations with lidocaine hydrochloride, and the dependence of lidocaine flux on the dosed amount of formulation.

TABLE 13

| | Formulation name | | | | | | | | |
|---|---|---|---|---|---|---|---|---|---|
| | Lidoderm ® | TC76 | TC79 | TC80 | TC81 | TC76a | TC79a | TC80a | TC81a |
| | Dosing (μl) | | | | | | | | |
| | | 3.0 | 3.0 | 3.0 | 3.0 | 10.0 | 10.0 | 10.0 | 10.0 |
| Percentages in | wt/wt % | wt/wt % | wt/wt % | wt/wt % | wt/wt % | wt/wt % | wt/wt % | wt/wt % | wt/wt % |
| Water | | 68.2 | 68.2 | 68.2 | 68.2 | 68.2 | 68.2 | 68.2 | 68.2 |
| D-Panthenol | | 5.8 | 11.6 | | 5.8 | 5.8 | 11.6 | | 5.8 |
| Propyl paraben | | 0.1 | 0.1 | 0.1 | 0.1 | 0.1 | 0.1 | 0.1 | 0.1 |
| Transcutol | | 5.8 | | 11.6 | 5.8 | 5.8 | | 11.6 | 5.8 |
| Tween 20 | | | 8 | 8 | 8 | | 8 | 8 | 8 |
| Lidocaine HCl | | 10 | 10 | 10 | 10 | 10 | 10 | 10 | 10 |
| monohydrate | | | | | | | | | |
| Methyl paraben | | 0.1 | 0.1 | 0.1 | 0.1 | 0.1 | 0.1 | 0.1 | 0.1 |
| Ethyl acetate | | 2 | 2 | 2 | 2 | 2 | 2 | 2 | 2 |

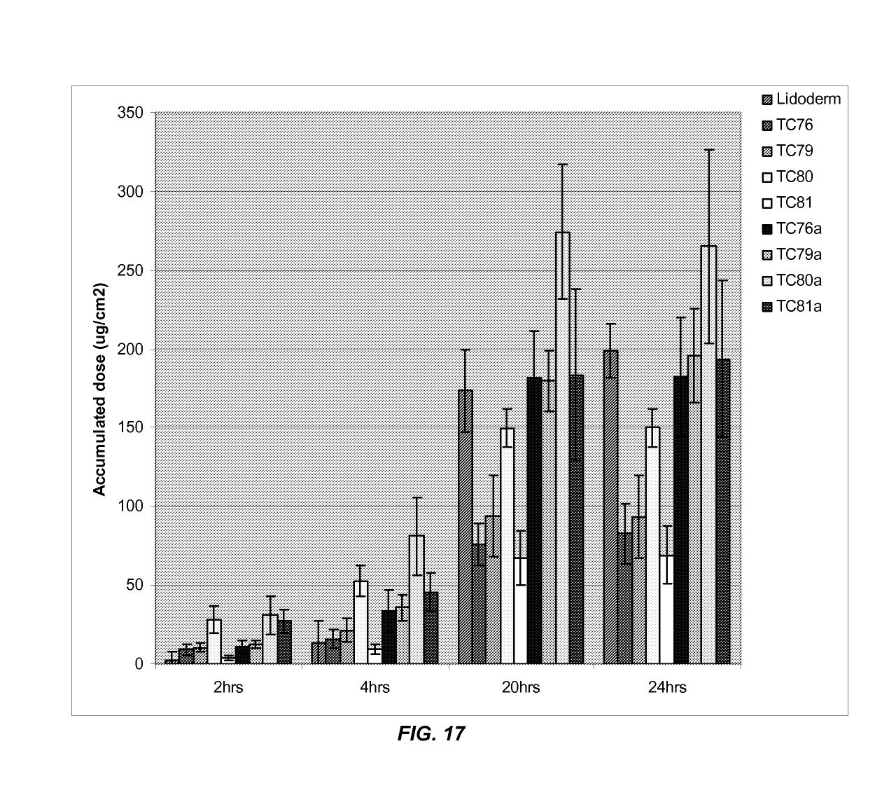

The results are shown in FIG. 17. The results indicate that the extent of lidocaine delivery scales with the amount of formulation applied.

### Example 13

This Example illustrates the use of triethyl citrate in formulations with lidocaine hydrochloride, and the dependence of lidocaine flux on the concentration of lidocaine hydrochloride in the formulation.

TABLE 14

| | Formulation name | | | | | | | | |
|---|---|---|---|---|---|---|---|---|---|
| | Lidoderm ® | TC82 | TC83 | TC84 | TC85 | TC86 | TC87 | TC88 | TC89 |
| | Dosing (μl) | | | | | | | | |
| | | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % |
| Triethyl citrate | | 2 | 2 | 2 | 2 | 2 | 2 | 2 | 2 |
| Tween 80 | | 8 | | | | 8 | | | |
| Tween 20 | | | 8 | 8 | 8 | | 8 | 8 | 8 |
| Transcutol | | 5.8 | | 11.6 | 5.8 | 5.8 | | 11.6 | 5.8 |
| D-Panthenol | | 5.8 | 11.6 | | 5.8 | 5.8 | 11.6 | | 5.8 |
| Water | | 66.2 | 66.2 | 66.2 | 66.2 | 61.2 | 61.2 | 61.2 | 61.2 |
| Propyl paraben | | 0.1 | 0.1 | 0.1 | 0.1 | 0.1 | 0.1 | 0.1 | 0.1 |
| Methyl paraben | | 0.1 | 0.1 | 0.1 | 0.1 | 0.1 | 0.1 | 0.1 | 0.1 |
| Lidocaine HCl | | 10 | 10 | 10 | 10 | 15 | 15 | 15 | 15 |
| monohydrate | | | | | | | | | |
| Thickener | | HY117 | HY117 | HY117 | HY117 | HY117 | HY117 | HY117 | HY117 |
| Wt % thickener | | 2 | 2 | 2 | 2 | 2 | 2 | 2 | 2 |
| added | | | | | | | | | |

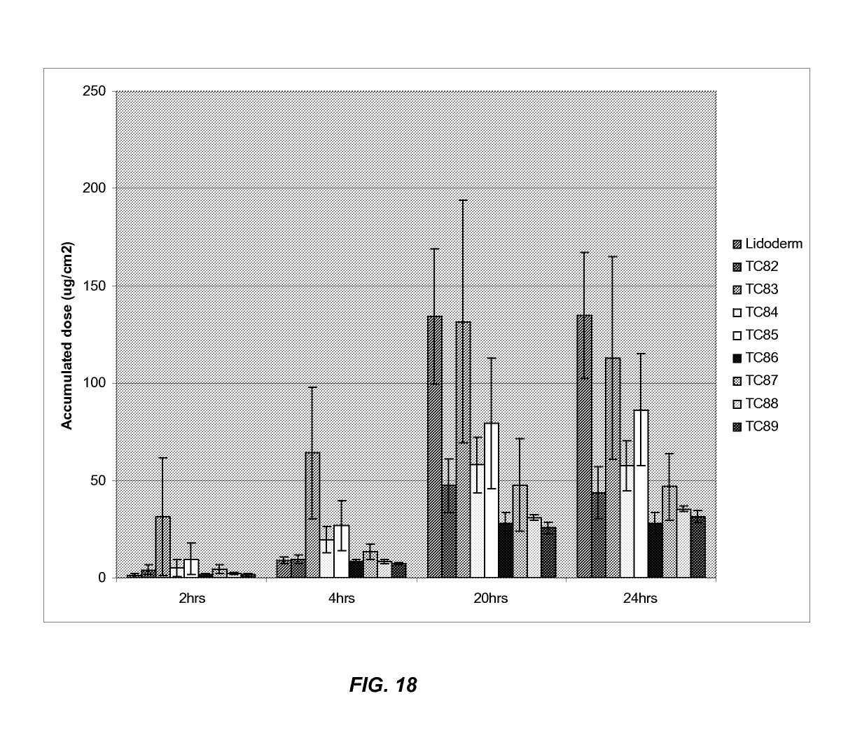

The permeation profile results are shown in FIG. 18. The results indicate that incorporation of a greater concentration of lidocaine hydrochloride formulation does not significantly enhance lidocaine transdermal flux.

### Example 14

This Example illustrates the use of triethyl citrate in formulations with lidocaine hydrochloride.

TABLE 15

| | Formulation name | | | | | | | | | |
|---|---|---|---|---|---|---|---|---|---|---|
| | Lidoderm | TC93 | TC94 | TC95 | TC96 | TC97 | TC98 | TC99 | TC100 | TC101 |
| | Dosing (μl) | | | | | | | | | |
| | | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % |
| Tween 20 | | 8.1 | 8.1 | 8.1 | 8.1 | 8.1 | 8.1 | 8.1 | 8.1 | 8.1 |
| D-Panthenol | | 6 | 9 | 3 | 6 | 9 | 3 | 3 | 3 | 3 |
| Transcutol | | 3 | | 6 | 3 | | 6 | 6 | 6 | 6 |
| Triethyl Citrate | | 3 | 3 | 3 | 3 | 3 | 3 | 3 | 3 | 3 |
| Lactic acid | | | | | | | 2 | 2 | 2 | 2 |
| Water | | 59.7 | 59.7 | 64.7 | 64.7 | 64.7 | 67.7 | 62.7 | 57.7 | 57.7 |
| Propyl paraben | | 0.1 | 0.1 | 0.1 | 0.1 | 0.1 | 0.1 | 0.1 | 0.1 | 0.1 |
| Methyl paraben | | 0.1 | 0.1 | 0.1 | 0.1 | 0.1 | 0.1 | 0.1 | 0.1 | 0.1 |
| Lidocaine HCl | | 20 | 20 | 15 | 15 | 15 | 10 | 15 | 20 | 15 |
| monohydrate | | | | | | | | | | |

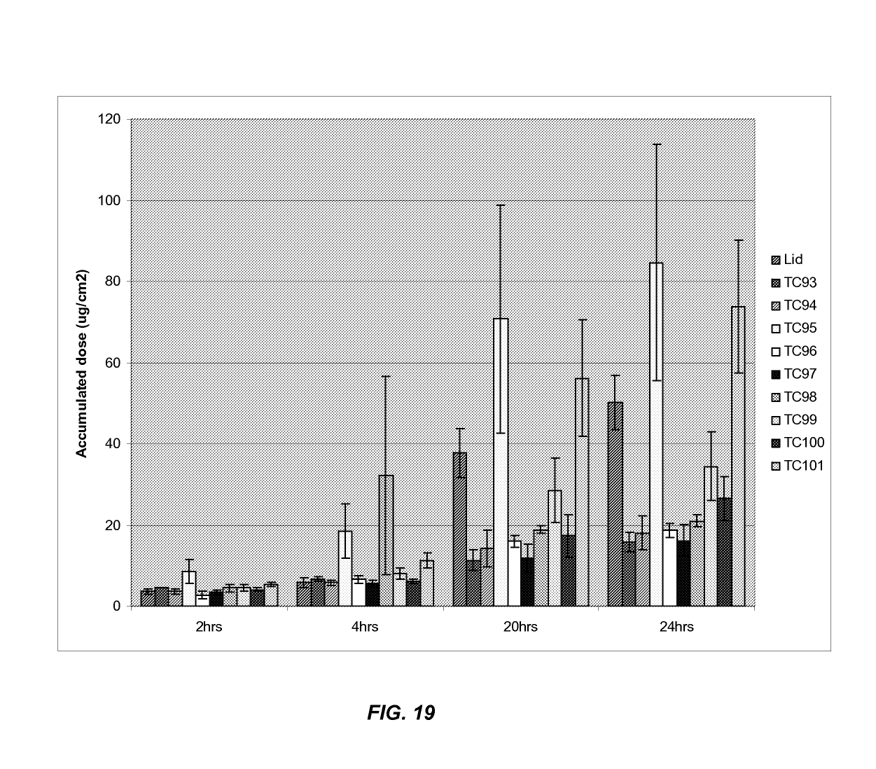

The permeation profile results are shown in FIG. 19. The results confirm the results provided in earlier examples. Importantly, and unexpectedly, it is discovered that providing a mixture of lidocaine base and lidocaine hydrochloride monohydrate in the formulation is more effective at enhancing lidocaine permeation than provision of lidocaine only as the lidocaine hydrochloride monohydrate salt (data for TC 100 compared with those for TC 101).

From FIG. 19 it can be seen that the TC 95 formulation with 15% lidocaine hydrochloride monohydrate shows remarkable lidocaine permeation enhancement relative to the TC 100 counterpart (with 20% lidocaine hydrochloride monohydrate) and relative to all other formulations in the TC93-TC 101 group, except TC 101.

### Example 15

This Example illustrates the use of triethyl citrate in formulations with lidocaine hydrochloride, in combination with D-panthenol and/or transcutol. This example further illustrates the effects of changing the concentration of lidocaine in the inventive formulations.

TABLE 16

| | Formulation name | | | | | | | | |
|---|---|---|---|---|---|---|---|---|---|
| | Lidoderm ® | TC102 | TC103 | TC104 | TC105 | TC106 | TC107 | TC109 | TC110 |
| | Dosing (μl) | | | | | | | | |
| | | 3 | 3 | 3 | 3 | 3 | 3 | 3 | 3 |
| Percentages in | wt/wt % | wt/wt % | wt/wt % | wt/wt % | wt/wt % | wt/wt % | wt/wt % | wt/wt % | wt/wt % |
| Tween 20 | | 8.1 | 8.1 | 8.1 | 8.1 | 8.1 | 8.1 | 8.1 | 8.1 |
| D-Panthenol | | 3 | 6 | 9 | 3 | 6 | 9 | 3 | 3 |
| Transcutol | | 6 | 3 | | 6 | 3 | | 6 | 6 |
| Triethyl Citrate | | 3 | 3 | 3 | 3 | 3 | 3 | 3 | 3 |
| Lactic acid | | | | | | | | 2 | 2 |
| Buffer pH 5.5 | | 59.7 | 59.7 | 59.7 | 64.7 | 64.7 | 64.7 | 57.7 | 57.7 |
| Propyl paraben | | 0.1 | 0.1 | 0.1 | 0.1 | 0.1 | 0.1 | 0.1 | 0.1 |
| Methyl paraben | | 0.1 | 0.1 | 0.1 | 0.1 | 0.1 | 0.1 | 0.1 | 0.1 |
| Lidocaine HCl | | 20 | 20 | 20 | 15 | 15 | 15 | 20 | 15 |
| monohydrate | | | | | | | | | |
| Lidocaine base | 5 | | | | | | | | 5 |

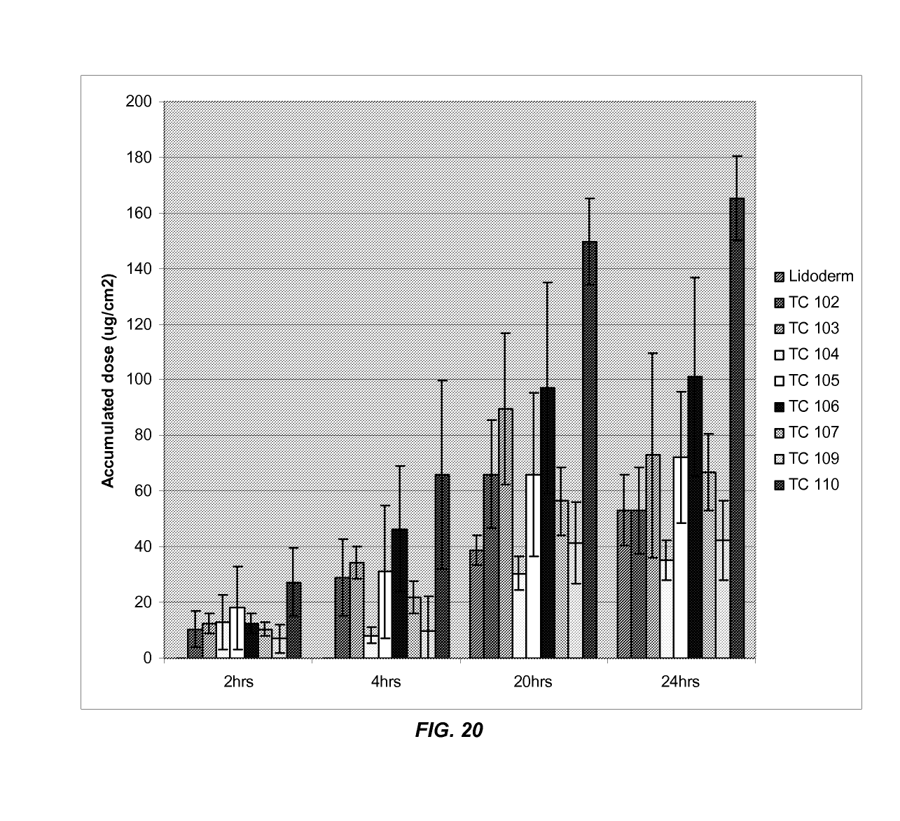

The permeation profile results are shown in FIG. 20.
Notably, formulations with 15% lidocaine hydrochloride monohydrate (TC105-107) evidence lidocaine fluxes comparable to those obtained with similar formulations containing 20% lidocaine hydrochloride monohydrate (TC102-104).

At higher lidocaine loading levels, lidocaine delivery is again enhanced when a mixture of both lidocaine hydrochloride monohydrate and lidocaine base at (3:1) ratio is used (TC110 compared with TC109 in FIG. 20 and TC 100 compared with TC 101 in FIG. 19). FIG. 20 evidences that the TC110 inventive formulation exhibits approximately 4 times higher lidocaine permeation than Lidoderm® at 22 and 24 hr periods. The replacement of water with a pH 5.5 aqueous buffer not only does not reduce the lidocaine permeation but actually enhances it significantly (for example, compare TC93-TC101 (FIG. 19) versus TC102-TC110 (FIG. 20).

### Example 16

This Example illustrates the use of thickeners in combination with triethyl citrate in formulations with lidocaine hydrochloride.

TABLE 17

| | Formulation name | | | | | | | | | |
|---|---|---|---|---|---|---|---|---|---|---|
| | TC53 | PVP_1% | PVP_0.5% | HY117_1% | HY117_0.5% | Nat_0.5% | Xan_0.2% | Xan_0.1% | PV90_0.2 | HPMC_0.2 |
| | Dosing (μl) | | | | | | | | | |
| | 3.0 | 3.0 | 3.0 | 3.0 | 3.0 | 3.0 | 3.0 | 3.0 | 3.0 | 3.0 |
| Percentages in | wt/wt % | wt/wt % | wt/wt % | wt/wt % | wt/wt % | wt/wt % | wt/wt % | wt/wt % | wt/wt % | wt/wt % |
| Triethyl Citrate | 2 | 2 | 2 | 2 | 2 | 2 | 2 | 2 | 2 | 2 |
| Tween 20 | 8.1 | 8.1 | 8.1 | 8.1 | 8.1 | 8.1 | 8.1 | 8.1 | 8.1 | 8.1 |
| Transcutol | 8.5 | 8.5 | 8.5 | 8.5 | 8.5 | 8.5 | 8.5 | 8.5 | 8.5 | 8.5 |
| D-Panthenol | 3 | 3 | 3 | 3 | 3 | 3 | 3 | 3 | 3 | 3 |
| Water | 68.2 | 68.2 | 68.2 | 68.2 | 68.2 | 68.2 | 68.2 | 68.2 | 68.2 | 68.2 |
| Propyl Paraben | 0.1 | 0.1 | 0.1 | 0.1 | 0.1 | 0.1 | 0.1 | 0.1 | 0.1 | 0.1 |
| Methyl Paraben | 0.1 | 0.1 | 0.1 | 0.1 | 0.1 | 0.1 | 0.1 | 0.1 | 0.1 | 0.1 |
| Lidocaine HCl | 10 | 10 | 10 | 10 | 10 | 10 | 10 | 10 | 10 | 10 |
| monohydrate | | | | | | | | | | |

The following abbreviations are used in Table 17.
PVP: Polyvinyl pyrrolidone K 30;
HY 117: Hydroxypropyl cellulose low viscosity (75-150 centipoise);
Nat (Natrosol): Hydroxyethyl cellulose;
Xan: Xanthan Gum;
PV90: Polyvinyl pyrrolidone K 90;
HPMC: Hydroxymethyl cellulose.

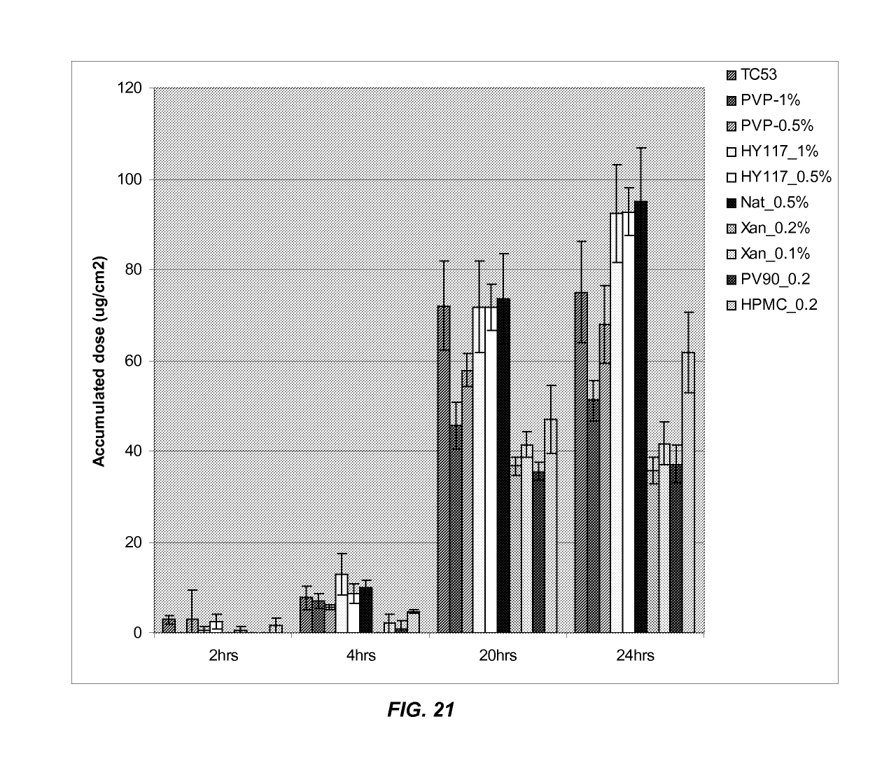

For each of the compositions provided in Table 17, the TC53 composition was first prepared, according to the weight percentages in Table 17 and then thickening agent was added to the weight percentage provided in the Formulation Name row of Table 17 (the slight change in weight percentage of each of the formulation constituents other than the thickener is not included in the Table 17). The permeation profile results are shown in FIG. 21. The results indicate that a variety of thickening agents can be incorporated into the current inventive formulations, without significant loss of performance. The addition of hydroxypropyl cellulose at each of 0.5 and 1% weight percent and hydroxyethyl cellulose at 0.5% weight percent do not reduce the lidocaine permeation; these particular thickening agents in fact provides a slight enhancement in lidocaine flux at 24 hours.

### Example 17

The following example illustrates a preferred embodiment of the present invention.

A. Features of Certain Formulations of the Present Invention.
0.1-20% of a topically acting anesthetic active ingredient, preferably lidocaine HCl monohydrate;
0.01-20% a pharmaceutically acceptable short-chain or branched alkyl ester up to C10, or combinations thereof, such as citric ester; preferably as triethyl citrate;
2-10% of a pharmaceutically acceptable surfactant; preferably nonionic surfactant; more preferably Tween 80 or Tween 20;
5-25% of a pharmaceutically acceptable co-surfactant, such as a water soluble solvent or combination thereof; preferably an alcohol or polyol, or combination thereof; more preferably propylene glycol, transcutol, PEG300, glycerine, transcutol, butanediol, hexanediol, panthenol, or combinations thereof;
30-80% water; and
0-0.5% of a preservative; such as a pharmaceutically acceptable antimicrobial; preferably methyl paraben or propyl paraben, or combinations thereof.

B. General Description of the Preparation of a Typical Triethyl Citrate Based Formulation (TC53)

### Composition:

Lidocaine HCl monohydrate: 10%
Triethyl citrate: 2%

### Transcutol: 8.5%

### Polysorbate 20 (Tween 20): 8.1%

### D-Panthenol: 3%

### Water: 68.2%

Methyl paraben: 0.1%
Propyl paraben: 0.1%

Procedure:

1—Combine D-panthenol and transcutol and vortex until a clear solution is obtained.
2—Add polysorbate 20, triethyl citrate and water and vortex.
3—Add Propyl paraben and Methyl paraben and vortex.
4—Add lidocaine HCl monohydrate and vortex (and warming briefly to 60-70° C. if necessary) to obtain a homogeneous solution.

### Example 18

This Example describes the short-term physical, microbiological and chemical stability of a prototype formulation for a period of three months under long-term (i.e., 25±2° C., 60%±5% RH) conditions.

### Equipment & Materials

The TC53 formulation, of composition as provided in Table 18, was manufactured at Nuvo Manufacturing facility (Varennes, Quebec) at a scale of 6 kg, and placed in stability chambers at appropriate temperatures in vials kept at horizontal orientation.

TABLE 18

| Ingredient | TC53 (%, wt/wt) |
|---|---|
| Water | 68.2 (q.s.) |
| Panthenol (racemic) | 3.0 |
| Triethyl citrate | 2.0 |
| Transcutol | 8.5 |
| Tween 20 | 8.1 |
| Methyl paraben | 0.1 |
| Propyl paraben | 0.1 |
| Lidocaine hydrochloride | 10.0 |
| monohydrate | |

### Test Methods

HPLC methods are used for the lidocaine HCl monohydrate assay, identification, and impurities profile of TC53. The other methods are compendial.

TABLE 19A
Specifications for TC53-Racemic

| TESTS | LIMITS | METHODS |
|---|---|---|
| Description | Clear, colorless liquid with a | Visual |
| | faint odor, essentially free of | |
| | visible foreign matter and | |
| | crystallized particles | |
| Lidocaine HCl Assay | 9.0-11.0% w/w | HPLC |
| | (10.0% w/w as label claim) | |
| pH | To report | Compendial |
| Total Impurity | NMT 1% | HPLC |
| Total Aerobic Microbial | ≦100 CFU/mL | Compendial |
| Count | | |
| Combined Yeasts and | ≦10 CFU/mL | Compendial |
| Molds | | |

### Stability Program and Storage Conditions

The stability of product was evaluated at 25° C. ±2° C./60%±5% RH and at 40° C.±2° C./75%±5% RH, with bottles being placed in horizontal position for 0, 1, 2, and 3 months.

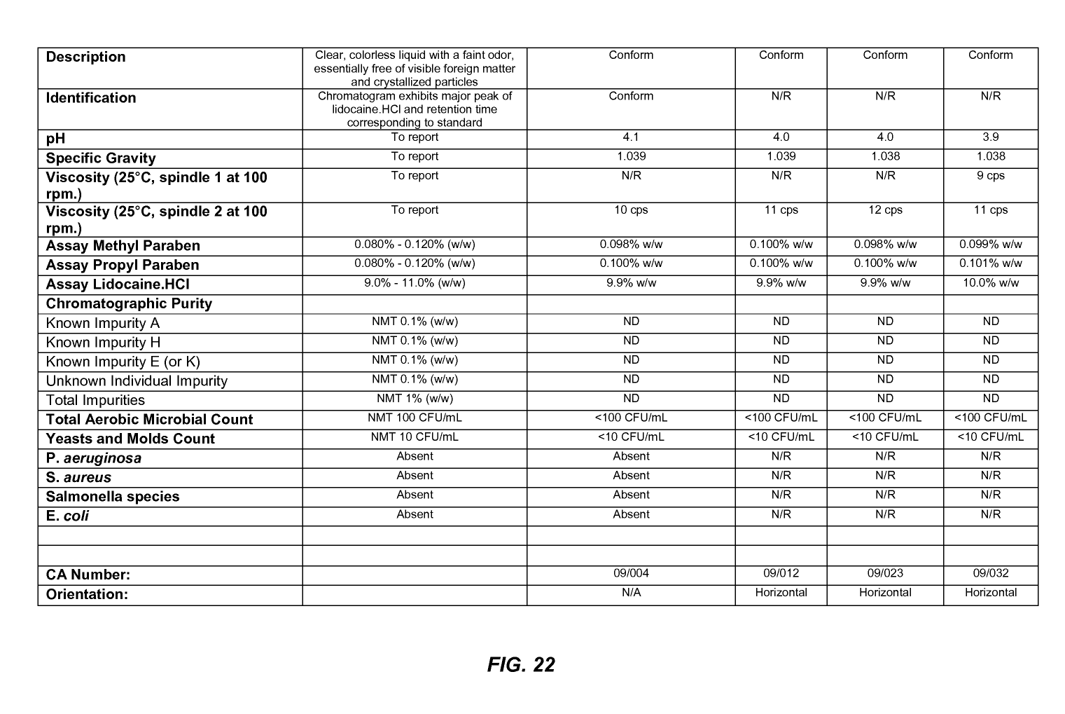

The results of the stability studies for this preferred embodiment is presented in Tables 19B and 19C as well as FIG. 22. The pH of the formulation appeared to decrease slightly over the course of the test, but the formulation was otherwise unchanged.

TABLE 19B
Three-Month Stability Study of Lidocaine HCl FormulationNRI-ANA-08 (TC53-Racemic)) at 25° C.

| Tests | Methods | Limit | Initial | 1 month | 2 month | 3 month |
|---|---|---|---|---|---|---|
| Description | Visual | Clear, colorless | Conform | Conform | Conform | Conform |
| | | liquid with a | | | | |
| | | faint odor, | | | | |
| | | essentially free | | | | |
| | | of visible | | | | |
| | | foreign matter | | | | |
| | | and crystallized | | | | |
| | | particles | | | | |
| Assay | HPLC | 9.0%-11.0% | 9.9% | 9.9% | 9.9% | 10.0% |
| Lidocaine | | (w/w) | (w/w) | (w/w) | (w/w) | (w/w) |
| HCl | | | | | | |
| Total | HPLC | NMT 1% | N/D | N/D | N/D | N/D |
| Impurities | | (w/w) | | | | |
| pH | Compendial | To report | 4.1 | 4.0 | 4.0 | 3.9 |
| Total | Compendial | NMT 100 CFU/mL | <100 CFU/mL | <100 CFU/mL | <100 CFU/mL | <100 CFU/mL |
| Aerobic | | | | | | |
| Microbial | | | | | | |
| Count | | | | | | |
| Yeasts and | Compendial | NMT 10 CFU/mL | <10 CFU/mL | <10 CFU/mL | <10 CFU/mL | <10 CFU/mL |
| Molds | | | | | | |
| Count | | | | | | |

TABLE 19C
Three-Month Stability Study of Lidocaine HCl Formulation NRI-ANA-08 (TC53-Racemic)) at 40° C.

| Tests | Methods | Limit | Initial | 1 month | 2 month | 3 month |
|---|---|---|---|---|---|---|
| Description | Visual | Clear, colorless | Conform | Conform | Conform | Conform |
| | | liquid with a | | | | |
| | | faint odor, | | | | |
| | | essentially free | | | | |
| | | of visible | | | | |
| | | foreign matter | | | | |
| | | and crystallized | | | | |
| | | particles | | | | |
| Assay | HPLC | 9.0%-11.0% | 9.9% w/w | 9.8% w/w | 9.9% w/w | 9.9% w/w |
| Lidocaine | | (w/w) | | | | |
| HCl | | | | | | |
| Total | HPLC | NMT 1% | N/D | N/D | N/D | ND |
| Impurities | | (w/w) | | | | |
| pH | Compendial | To report | 4.1 | 4.1 | 4.4 | 4.4 |
| Total | Compendial | NMT 100 CFU/mL | <100 CFU/mL | <100 CFU/mL | <100 CFU/mL | <100 CFU/mL |
| Aerobic | | | | | | |
| Microbial | | | | | | |
| Count | | | | | | |
| Yeasts and | Compendial | NMT 10 CFU/mL | <10 CFU/mL | <10 CFU/mL | <10 CFU/mL | <10 CFU/mL |
| Molds | | | | | | |
| Count | | | | | | |

### Example 19

This Example illustrates the use of triethyl citrate in a formulation with lidocaine hydrochloride, with or without D-panthenol, compared with Lidoderm®.

TABLE 20

| | | TC113 |
|---|---|---|
| Formulation name | TC53-D | (NRI-ANA-14) |
| Dosing (μl) | 3.0 | 3.0 |
| Percentages in | wt/wt % | wt/wt % |
| Triethyl Citrate | 2 | 2 |
| Tween 20 | 8.1 | 8.1 |
| Transcutol | 8.5 | 8.5 |
| D-Panthenol | 3 | |
| Water | 68.2 | 71.2 |
| Propyl Paraben | 0.1 | 0.1 |
| Methyl Paraben | 0.1 | 0.1 |
| Lidocaine HCl | 10 | 10 |
| monohydrate | | |

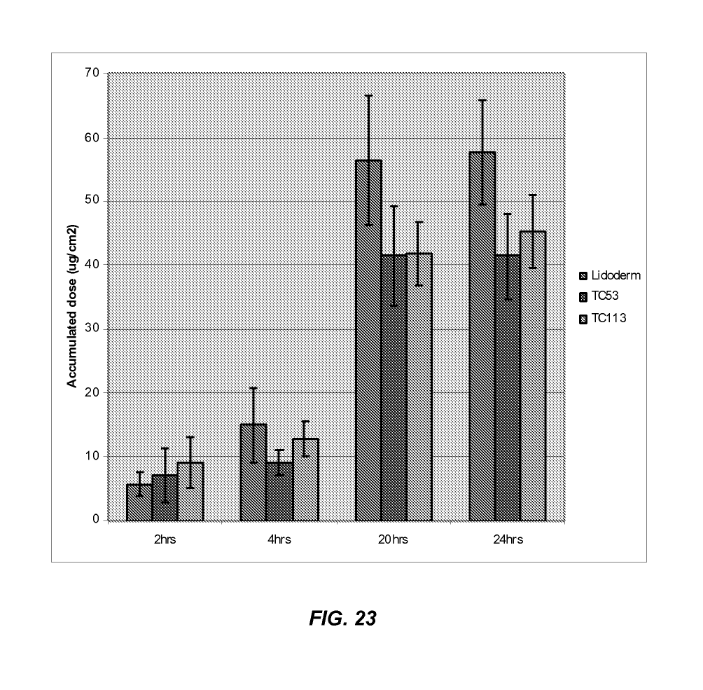

The permeation profile results are shown in FIG. 23. The formulations with and without D-panthenol have a similar permeation profile.

### Example 20

This Example provides the results of stability tests on three exemplary formulations.

TABLE 21
Composition of 10% Lidocaine Formulations (NRI-ANA)

| | | | | % | | |
|---|---|---|---|---|---|---|
| | | | | | NRI- | NRI- |
| | | | | | ANA- | ANA- |
| | | | | NRI-ANA-08 | 13 | 14 |
| Component | Chemical | | | (TC53- | (TC53- | (TC- |
| (CAS#) | Name | Chemical Structure | Function | Racemic) | D) | 113) |
| Lidocaine HCl, USP (137-58-6) | 2- (Diethyl- amino)-N-(2,5 dimethyl- phenyl) acetamide, hydrochloride | 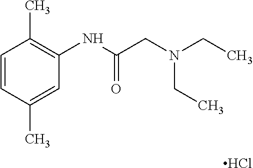 | Active | 10.0 | | |
| D,L-Panthenol, USP (16485-10- 2) or Dexpanthenol, USP (81-13-0)¹ | (2R)-2,4- dihydroxy-N- (3- hydroxy- propyl)-2,3,3- trimethyl- butanamide (D- enantiomer) | 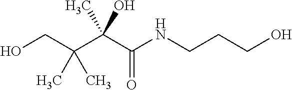 | Solvent | 1.5² | 3.0³ | — |
| | (2S)-2,4- dihydroxy-N- (3 hydroxy- propyl)-2,3,3 trimethyl- butanamide (L- enantiomer | 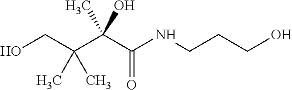 | Solvent | 1.5² | — | — |

| | | | | |
|---|---|---|---|---|
| Triethyl citrate, NF (77-93-0) | Diethyl 2-(2- ethoxy-2- hydroxyethyl)- 2- hydroxy- butanedicate | 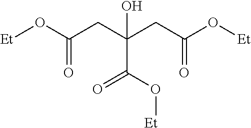 | Enhancer | 20 |
| Polysorbate 20, NF (9005-64-5) | Sorbitan 20, Tween 20, ethoxylated sorbitan monolaurate | 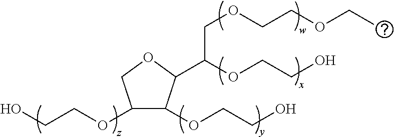 | Thickening agent | 8.1 |
| Diethylene Glycol Monoethyl Ether, NF (111- 90-0) | Transcutol 2- (2- ethoxyethoxy) ethanol | 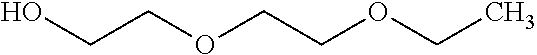 | Solvent | 8.5 |
| Methylparaben, NF (99-76-3) | Propyl 4- hydroxy- benzoate | 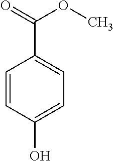 | Preserva- tive | 0.1 |
| Propylparaben, NF (94-13-3) | Methyl 4- hydroxy- benzoate | 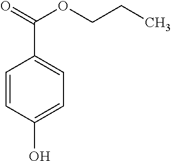 | Preserva- tive | 0.1 |
| Water | Water | H₂O | Solvent | qs |

¹Note:
D,L-panthenol is also listed under other CAS#s
²Formulation NRI-ANA-08 contains 3% racemic mixture panthenol
³Formulation NRI-ANA-13 contains 3% of the D-enantiomer of panthenol (dexpanthenol)

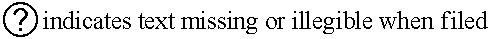

### Stability Program and Storage Conditions

As in Example 18, the stability of product was evaluated at 25° C. ±2° C./60%±5% RH and at 40° C. ±2° C./75%±5% RH, with bottles being placed in horizontal position for 0, 1, 2, and 3 months.

The results of the stability studies for this preferred embodiment is presented in Tables 22A-F.

TABLE 22A
Three-Month Stability Study of Lidocaine HCl FormulationNRI-ANA-08 (25° C.)

| Tests | Methods | Limit | Initial | 1 month | 2 month | 3 month |
|---|---|---|---|---|---|---|
| Description | Visual | Clear, | Conform | Conform | Conform | Conform |
| | | colorless | | | | |
| | | liquid with a | | | | |
| | | faint odor, | | | | |
| | | essentially | | | | |
| | | free of visible | | | | |
| | | foreign matter | | | | |
| | | and | | | | |
| | | crystallized | | | | |
| | | particles | | | | |
| Assay | HPLC | 9.0%-11.0% | 10% | 9.9% | 9.9% | 10.0% |
| Lidocaine | | (w/w) | | | | |
| HCl | | | | | | |
| Total | HPLC | NMT 1% | N/D | N/D | N/D | <LOQ |
| Impurities | | (w/w) | | | | |
| pH | Compendial | To report | 4.56 | 4.41 | 4.58 | 4.40 |
| Total | Compendial | NMT 100 CFU/mL | <50 cfu/mL | <50 cfu/mL | <50 cfu/mL | <50 cfu/mL |
| Aerobic | | | | | | |
| Microbial | | | | | | |
| Count | | | | | | |
| Yeasts and | Compendial | NMT 10 CFU/mL | <5 cfu/mL | <5 cfu/mL | <5 cfu/mL | <5 cfu/mL |
| Molds Count | | | | | | |

TABLE 22B
Three-Month Stability Study of Lidocaine HCl FormulationNRI-ANA-08
(40° C.)

| Tests | Methods | Limit | Initial | 1 month | 2 month | 3 month |
|---|---|---|---|---|---|---|
| Description | Visual | Clear, | Conform | Conform | Conform | Conform |
| | | colorless | | | | |
| | | liquid with | | | | |
| | | a faint odor, | | | | |
| | | essentially | | | | |
| | | free of | | | | |
| | | visible | | | | |
| | | foreign | | | | |
| | | matter and | | | | |
| | | crystallized | | | | |
| | | particles | | | | |
| Assay | HPLC | 9.0%-11.0% | 10% | 9.9% | 9.9% | 10.0% |
| Lidocaine | | (w/w) | | | | |
| HCl | | | | | | |
| Total | HPLC | NMT 1% | N/D | N/D | N/D | <LOQ |
| Impurities | | (w/w) | | | | |
| pH | Compendial | To report | 4.56 | 4.65 | 4.84 | 4.66 |
| Total | Compendial | NMT 100 CFU/mL | <50 cfu/mL | <50 cfu/mL | <50 cfu/mL | <50 cfu/mL |
| Aerobic | | | | | | |
| Microbial | | | | | | |
| Count | | | | | | |
| Yeasts and | Compendial | NMT 10 CFU/mL | <5 cfu/mL | <5 cfu/mL | <5 cfu/mL | <5 cfu/mL |
| Molds | | | | | | |
| Count | | | | | | |

TABLE 22C
Three-Month Stability Study of Lidocaine HCl FormulationNRI-ANA-13
(25° C.)

| Tests | Methods | Limit | Initial | 1 month | 2 month | 3 month |
|---|---|---|---|---|---|---|
| Description | Visual | Clear, colorless | Conform | Conform | Conform | Conform |
| | | liquid with a | | | | |
| | | faint odor, | | | | |
| | | essentially free | | | | |
| | | of visible | | | | |
| | | foreign matter | | | | |
| | | and | | | | |
| | | crystallized | | | | |
| | | particles | | | | |
| Assay | HPLC | 9.0%-11.0% | 10% | 9.9% | 9.9% | 10.0% |
| Lidocaine HCl | | (w/w) | | | | |
| Total Impurities | HPLC | NMT 1% | N/D | N/D | N/D | <LOQ |
| | | (w/w) | | | | |
| pH | Compendial | To report | 4.82 | 4.73 | 4.82 | 4.67 |
| Total Aerobic | Compendial | NMT 100 CFU/mL | <50 cfu/mL | <50 cfu/mL | <50 cfu/mL | <50 cfu/mL |
| Microbial Count | | | | | | |
| Yeasts and | Compendial | NMT 10 CFU/mL | <5 cfu/mL | <5 cfu/mL | <5 cfu/mL | <5 cfu/mL |
| Molds Count | | | | | | |

TABLE 22D
Three-Month Stability Study of Lidocaine HCl FormulationNRI-ANA-13
(40° C.)

| Tests | Methods | Limit | Initial | 1 month | 2 month | 3 month |
|---|---|---|---|---|---|---|
| Description | Visual | Clear, colorless | Conform | Conform | Conform | Conform |
| | | liquid with a | | | | |
| | | faint odor, | | | | |
| | | essentially free | | | | |
| | | of visible foreign | | | | |
| | | matter and | | | | |
| | | crystallized | | | | |
| | | particles | | | | |
| Assay | HPLC | 9.0%-11.0% | 10% | 9.9% | 9.9% | 10.0% |
| Lidocaine•HCl | | (w/w) | | | | |
| Total | HPLC | NMT 1% (w/w) | N/D | N/D | N/D | <LOQ |
| Impurities | | | | | | |
| pH | Compendial | To report | 4.82 | 4.83 | 4.99 | 4.82 |
| Total Aerobic | Compendial | NMT 100 CFU/mL | <50 cfu/mL | <50 cfu/mL | <50 cfu/mL | <50 cfu/mL |
| Microbial | | | | | | |
| Count | | | | | | |
| Yeasts and | Compendial | NMT 10 CFU/mL | <5 cfu/mL | <5 cfu/mL | <5 cfu/mL | <5 cfu/mL |
| Molds Count | | | | | | |

TABLE 22E
Three-Month Stability Study of Lidocaine HCl FormulationNRI-ANA-14
(25° C.)

| Tests | Methods | Limit | Initial | 1 month | 2 month | 3 month |
|---|---|---|---|---|---|---|
| Description | Visual | Clear, | Conform | Conform | Conform | Conform |
| | | colorless | | | | |
| | | liquid with | | | | |
| | | a faint | | | | |
| | | odor, | | | | |
| | | essentially | | | | |
| | | free of | | | | |
| | | visible | | | | |
| | | foreign | | | | |
| | | matter and | | | | |
| | | crystallized | | | | |
| | | particles | | | | |
| Assay | HPLC | 9.0%-11.0% | 10% | 9.9% | 9.9% | 10.0% |
| Lidocaine HCl | | (w/w) | | | | |
| Total | HPLC | NMT 1% | N/D | N/D | N/D | <LOQ |
| Impurities | | (w/w) | | | | |
| pH | Compendial | To report | 4.39 | 4.24 | 4.28 | 3.99 |
| Total Aerobic | Compendial | NMT 100 CFU/mL | <50 cfu/mL | <50 cfu/mL | <50 cfu/mL | <50 cfu/mL |
| Microbial | | | | | | |
| Count | | | | | | |
| Yeasts and | Compendial | NMT 10 CFU/mL | <5 cfu/mL | <5 cfu/mL | <5 cfu/mL | <5 cfu/mL |
| Molds Count | | | | | | |

TABLE 22F
Three-Month Stability Study of Lidocaine HCl FormulationNRI-ANA-14
(40° C.)

| Tests | Methods | Limit | Initial | 1 month | 2 month | 3 month |
|---|---|---|---|---|---|---|
| Description | Visual | Clear, | Conform | Conform | Conform | Conform |
| | | colorless | | | | |
| | | liquid with | | | | |
| | | a faint | | | | |
| | | odor, | | | | |
| | | essentially | | | | |
| | | free of | | | | |
| | | visible | | | | |
| | | foreign | | | | |
| | | matter and | | | | |
| | | crystallized | | | | |
| | | particles | | | | |
| Assay | HPLC | 9.0%-11.0% | 10% | 9.9% | 9.9% | 10.0% |
| Lidocaine HCl | | (w/w) | | | | |
| Total | HPLC | NMT 1% | N/D | <LOQ | <LOQ | <LOQ |
| Impurities | | (w/w) | | | | |
| pH | Compendial | To report | 4.39 | 3.9 | 3.65 | 3.21 |
| Total Aerobic | Compendial | NMT 100 CFU/mL | <50 cfu/mL | <50 cfu/mL | <50 cfu/mL | <50 cfu/mL |
| Microbial | | | | | | |
| Count | | | | | | |
| Yeasts and | Compendial | NMT 10 CFU/mL | <5 cfu/mL | <5 cfu/mL | <5 cfu/mL | <5 cfu/mL |
| Molds Count | | | | | | |

### Example 21

This Example provides the results of stability tests on the three exemplary formulations set forth in Example 20 using the procedure set forth in Example 20.

Formulation Stability over Time

The initial pH values of NRI-ANA-08 (D,L-panthenol) and NRI-ANA-14 (panthenol free) were 4.1 and 4.2, respectively, consistent with the theoretical value of 4.2 for a 10% lidocaine solution. In contrast to NRI-ANA-08 and NRI-ANA-14, the initial pH value of NRI-ANA-13 (D-panthenol) was 4.8.

The pH of NRI-ANA-14 decreased from an initial value of 4.2 to 2.6 after 6 months at 40° C. and to 3.5 at 25° C. The decrease in pH of NRI-ANA-14 was accompanied by a concomitant decrease in triethyl citrate (TEC) concentration from a nominal value of 2.0% to 1.3% after 6 months at 40° C.

### Proposed Cause for pH Change

Without being bound by any particular theory, the gradual decrease in pH for NRI-ANA-14 was proposed to result from hydrolysis of TEC producing acidic degradants such as diethyl citrates, ethyl citrates and citric acid.

A process impurity (and/or degradant) of panthenol, 3-aminopropanol (AMP), was proposed to cause the initial higher pH value of NRI-ANA-13 (pH 4.8 vs. theoretical value of pH 4.2). The stabilization of NRI-ANA-08 and NRI-ANA-13 by panthenol was believed to result from the presence of AMP, which would help to maintain the pH of the formulation.

### Investigation of the Cause

Experiment A: AMP (0.0006%) was added to NRI-ANA-14 to replicate the AMP concentration in NRI-ANA-08. After AMP addition, the pH of NRI-ANA-08 was 4.1, and the pH of NRI-ANA-14 (+0.0006% AMP) was 4.2. This supported the hypothesis.

Experiment B: AMP (0.012%) was added to NRI-ANA-14 to replicate the AMP concentration in NRI-ANA-13. After AMP addition, the pH of NRI-ANA-13 was 4.8, and the pH of NRI-ANA-14 (+0.012% AMP) was 4.8. This supported the hypothesis.

Experiment C: A modified NRI-ANA-14 formulation containing 1.3% TEC and citric acid equivalent to 0.7% TEC was prepared. A control NRI-ANA-14 formulation was subjected to a stability study according to the method set forth in Example 19. After six months at 40° C., the pH of the control NRI-ANA-14 formulation was 2.6, while the pH of the modified NRI-ANA-14 formulation was 1.6. This supported the hypothesis.

Experiment D: Stability studies of the solutions from Experiments A and B were performed. As shown below in Table 23, the results supported the hypothesis.

Experiment E: The pH of 10% aqueous solution of D,L- and D-panthenol were measured. The pH of 10% D,L-panthenol was 6.7, but the pH of 10% D-panthenol was 9.4. This supported the hypothesis.

Experiment F: Stability studies of the buffered formulation were conducted at elevated temperature (70° C.). As shown below in Tables 23A-F, the results supported the hypothesis.

TABLE 23A
pH Data I

| | | Start Date | | | | | |
|---|---|---|---|---|---|---|---|
| | | December 2008 | August 2009 | | January 2010 | | |
| | | Formula# | | | | | |
| | | 08 | 13 | 14 | 08 | 13 | 14 |
| | 0 | 4.1 | 4.77 | 4.15 | 4.56 | 4.82 | 4.39 |
| | 1 m | 4.0 | 5.02 | 4.24 | 4.41 | 4.73 | 4.24 |
| | 2 m | 4.0 | 4.96 | 4.01 | 4.58 | 4.82 | 4.28 |
| 25 C. | 3 m | 3.9 | 4.75 | 4.02 | 4.40 | 4.67 | 3.99 |
| | 6 m | NT | 4.33 | 3.51 | | | |
| | 11 m | NT | 4.15 | 2.93 | | | |
| | 15/16 m | 3.82 | NT | NT | | | |
| | 1 m | 4.1 | 4.85 | 3.74 | 4.65 | 4.83 | 3.90 |
| | 2 m | 4.4 | 4.73 | 3.29 | 4.84 | 4.99 | 3.65 |
| 40 C. | 3 m | 4.4 | 4.83 | 3.38 | 4.66 | 4.82 | 3.21 |
| | 6 m | NT | 4.46 | 2.55 | | | |
| | 11 m | NT | 4.17 | 1.77 | | | |
| | 15/16 m | 4.06 | NT | NT | | | |

TABLE 23B
pH Data II

| | | Start Date | | | | | | | | | | | | |
|---|---|---|---|---|---|---|---|---|---|---|---|---|---|---|
| | | May 2010 | | | | | | | | | | | | |
| | | Formula# | | | | | | | | | | | | |
| | | | | | | | | | | | | 14 | | |
| | | | | | 08 | 08 | 13 | 13 | 14 | 14 | 14 | 100 | 14 | 14 |
| | | 08 | 13 | 14 | 50 mM | 100 mM | 50 mM | 100 mM | 50 mM | 100 mM | 50 mM | mM | 0.0006% | 0.012% |
| Lot specifics | | N/A | | | Citrate | Citrate | Citrate | Citrate | Citrate | Citrate | Citrate* | Citrate* | 3-Amp | 3-Amp |
| | 0 | | | | | | | 3.89 | | 3.93 | 4.11 | 4.20 | | |
| | 1 m | | | | | | | 3.78 | | 3.71 | 4.20 | 4.06 | | |
| | 2 m | | | | | | | Dis- | | | Discontinued | | | |
| 25 C. | 3 m | | | | | | | continued | | | | | | |
| | 6 m | | | | | | | | | | | | | |
| | 11 m | | | | | | | | | | | | | |
| | 15/16 m | | | | | | | | | | | | | |
| | 1 m | | | | | | | 3.78 | | 3.70 | 3.98 | 4.08 | | |
| | 2 m | | | | | | | Dis- | | | Discontinued | | | |
| 40 C. | 3 m | | | | | | | continued | | | | | | |
| | 6 m | | | | | | | | | | | | | |
| | 11 m | | | | | | | | | | | | | |
| | 15/16 m | | | | | | | | | | | | | |
| | 0 | 3.92 | 4.41 | 3.89 | 3.75 | 3.76 | 3.86 | 3.89 | 3.84 | 3.93 | 4.11 | 4.20 | 4.20 | 4.75 |
| | 1 w | 4.84 | 4.80 | 2.66 | 4.00 | 4.08 | 4.08 | 4.12 | 3.75 | 3.78 | 3.99 | 4.04 | 2.71 | 3.67 |
| 70 C. | 2 w | 4.89 | 5.04 | 2.23 | 4.13 | 4.05 | 4.16 | 4.14 | 3.57 | 3.66 | 3.91 | 3.97 | 2.37 | 3.02 |
| | 3 w | 4.90 | 4.91 | 1.99 | 4.41 | 4.33 | 4.46 | 4.26 | 3.62 | 3.61 | 3.81 | 3.93 | 2.01 | 2.58 |
| | 1 m | 4.81 | 4.85 | 1.88 | 4.35 | 4.30 | 4.41 | 4.34 | 3.44 | 3.54 | 3.73 | 3.90 | 1.93 | 2.22 |

*Final pH adjusted to 4.2

TABLE 23C
TEC Results I

| | | Start Date | | | | | |
|---|---|---|---|---|---|---|---|
| | | December 2008 | August 2009 | | January 2010 | | |
| | | Formula# | | | | | |
| | | 08 | 13 | 14 | 08 | 13 | 14 |
| | 0 | | | | | | |
| | 1 m | | | | | | |
| | 2 m | | | | | | |
| 25 C. | 3 m | | | | | | |
| | 6 m | NT | 1.97 | 1.96 | | | |
| | 11 m | NT | 1.96 | 1.93 | | | |
| | 15/16 m | 2.04 | NT | NT | | | |
| | 1 m | | | | | | |
| | 2 m | | | | | | |
| 40 C. | 3 m | | | | | | |
| | 6 m | NT | 1.87 | 1.29 | | | |
| | 11 m | NT | 1.79 | 0.33 | | | |
| | 15/16 m | 1.77 | NT | NT | | | |

TABLE 23D
TEC Results II

| | | Start Date | | | | | | | | | | | | |
|---|---|---|---|---|---|---|---|---|---|---|---|---|---|---|
| | | May 2010 | | | | | | | | | | | | |
| | | Formula# | | | | | | | | | | | | |
| | | | | | | | | | | 14 | | 14 | | |
| | | | | | 08 | 08 | 13 | 13 | 14 | 100 | 14 | 100 | 14 | 14 |
| | | 08 | 13 | 14 | 50 mM | 100 mM | 50 mM | 100 mM | 50 mM | mM | 50 mM | mM | 0.0006% | 0.012% |
| Lot specifics | | N/A | | | Citrate | Citrate | Citrate | Citrate | Citrate | Citrate | Citrate* | Citrate* | 3-Amp | 3-Amp |
| | | | | | | | | 2.02 | | 2.01 | 2.03 | 2.03 | | |
| | 0 | | | | | | | 2.03 | | 2.04 | 2.04 | 2.03 | | |

| | | | |
|---|---|---|---|
| 1 m | | Discontinued | Discontinued |
| | 2 m | | |

| | | | | | | | | | | | | | | |
|---|---|---|---|---|---|---|---|---|---|---|---|---|---|---|
| 25 C. | 3 m | | | | | | | | | | | | | |
| | 6 m | | | | | | | | | | | | | |
| | 11 m | | | | | | | | | | | | | |
| | 15/16 m | | | | | | | | | | | | | |
| | 1 m | | | | | | | 2.01 | | 2.01 | 2.01 | 2.01 | | |
| | 2 m | | | | | | | | | | Discontinued | | | |
| 40 C. | 3 m | | | | | | | | | | | | | |
| | 6 m | | | | | | | | | | | | | |
| | 11 m | | | | | | | | | | | | | |
| | 15/16 m | | | | | | | | | | | | | |
| | 0 | 2.04 | 2.03 | 2.01 | 2.02 | 2.02 | 2.02 | 2.02 | 2.02 | 2.01 | 2.03 | 2.03 | 2.02 | 2.02 |
| | 12 | 1.99 | 1.98 | 1.90 | 1.95 | 1.94 | 1.95 | 1.95 | 1.94 | 1.95 | 1.95 | 1.93 | 1.90 | 1.96 |
| 70 C. | 2 w | 1.92 | 1.90 | 1.62 | 1.87 | 1.85 | 1.87 | 1.86 | 1.87 | 1.86 | 1.88 | 1.85 | 1.64 | 1.88 |
| | 3 w | 1.85 | 1.84 | 1.12 | 1.80 | 1.79 | 1.79 | 1.78 | 1.80 | 1.79 | 1.83 | 1.78 | 1.18 | 1.71 |
| | 1 m | 1.78 | 1.75 | 0.56 | 1.69 | 1.66 | 1.69 | 1.67 | 1.72 | 1.69 | 1.74 | 1.68 | 0.60 | 1.32 |

*Final pH adjusted to 4.2

TABLE 23E
Panthenol Results I

| | | Start Date | | | | | |
|---|---|---|---|---|---|---|---|
| | | December 2008 | August 2009 | | January 2010 | | |
| | | Formula# | | | | | |
| | | 08 | 13 | 14 | 08 | 13 | 14 |
| | 0 | | | | | | |
| | 1 m | | | | | | |
| | 2 m | | | | | | |
| 25 C. | 3 m | | | | | | |
| | 6 m | NT | NT | NA | | | |
| | 11 m | NT | 2.94 | NA | | | |
| | 15/16 m | 2.92 | NT | NA | | | |
| | 1 m | | | | | | |
| | 2 m | | | | | | |
| 40 C. | 3 m | | | | | | |
| | 6 m | NT | NT | NA | | | |
| | 11 m | NT | 2.85 | NA | | | |
| | 15/16 m | 2.66 | NT | NA | | | |

TABLE 23F
Panthenol Results II

| | | Start Date | | | | | | | | | | | | |
|---|---|---|---|---|---|---|---|---|---|---|---|---|---|---|
| | | May 2010 | | | | | | | | | | | | |
| | | Formula# | | | | | | | | | | | | |
| | | | | | | | | | | 14 | | 14 | | |
| | | | | | 08 | 08 | 13 | 13 | 14 | 100 | 14 | 100 | 14 | 14 |
| | | 08 | 13 | 14 | 50 mM | 100 mM | 50 mM | 100 mM | 50 mM | mM | 50 mM | mM | 0.0006% | 0.012% |
| Lot specifics | | N/A | | | Citrate | Citrate | Citrate | Citrate | Citrate | Citrate | Citrate* | Citrate* | 3-Amp | 3-Amp |
| | 0 | | | | | | | NT | | | | | | |
| | 1 m | | | | | | | 2.96 | | | | | | |

| | |
|---|---|
| 2 m | Discontinued |

| | | | | | | | | | | | | | | |
|---|---|---|---|---|---|---|---|---|---|---|---|---|---|---|
| 25 C. | 3 m | | | | | | | | | | | | | |
| | 6 m | | | | | | | | | | | | | |
| | 11 m | | | | | | | | | | | | | |
| | 15/16 m | | | | | | | | | | | | | |
| | 1 m | | | | | | | 2.91 | | | | | | |
| | 2 m | | | | | | | | | | | | | |
| 40 C. | 3 m | | | | | | | | | | | | | |
| | 6 m | | | | | | | | | | | | | |
| | 11 m | | | | | | | | | | | | | |
| | 15/16 m | | | | | | | | | | | | | |
| | 0 | 3.03 | 3.01 | NA | 3.03 | 3.03 | 2.99 | NT | NA | NA | NA | NA | NA | NA |
| | 1 w | 2.94 | 2.94 | NA | 2.84 | 2.76 | 2.82 | 2.75 | NA | NA | NA | NA | NA | NA |
| 70 C. | 2 w | 2.86 | 2.87 | NA | 2.69 | 2.54 | 2.68 | 2.55 | NA | NA | NA | NA | NA | NA |
| | 3 w | 2.79 | 2.79 | NA | 2.57 | 2.40 | 2.55 | 2.38 | NA | NA | NA | NA | NA | NA |
| | 1 m | 2.70 | 2.71 | NA | 2.41 | 2.19 | 2.42 | 2.19 | NA | NA | NA | NA | NA | NA |

*Final pH adjusted to 4.2

### Summary of Buffer Effects (70° C.)

Triethyl Citrate (TEC)

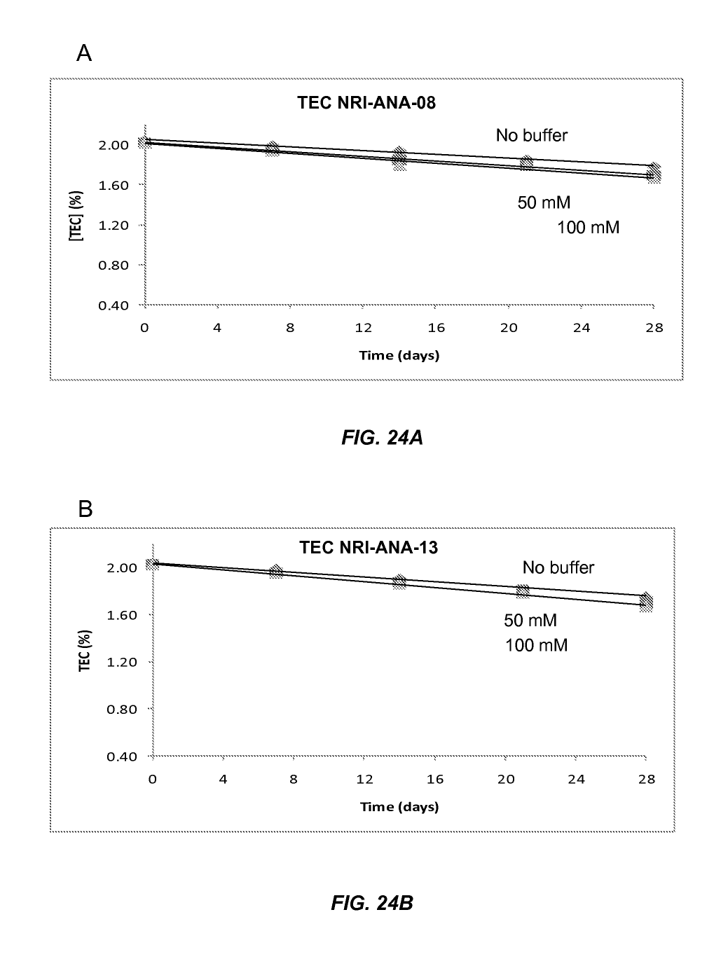

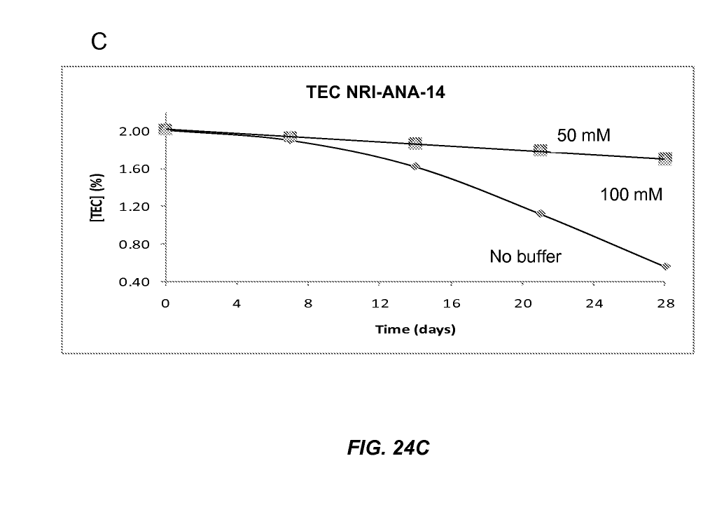

Buffering formulations NRI-ANA-08, NRI-ANA-13 at pH 3.8-3.9 with citrate helped to reduce the rate of pH change over time, but failed to reduce the rate of degradation of TEC, which was independent of citrate concentration. FIGS. 24 A-B show the effect of citrate buffer concentration on the degradation of triethyl citrate (TEC) in formulations NRI-ANA-08 and NRI-ANA-13.

Buffering formulation NRI-ANA-14 at pH 3.8-3.9 with citrate (50 or 100 mM) helped to reduce the pH change over time, and reduce the rate of degradation of TEC to the same rate as seen in NRI-ANA-08 and NRI-ANA-13. See FIG. 24C.

Panthenol (PAN)

In the absence of buffer, panthenol (PAN) and triethyl citrate (TEC) were found to degrade at similar rates.

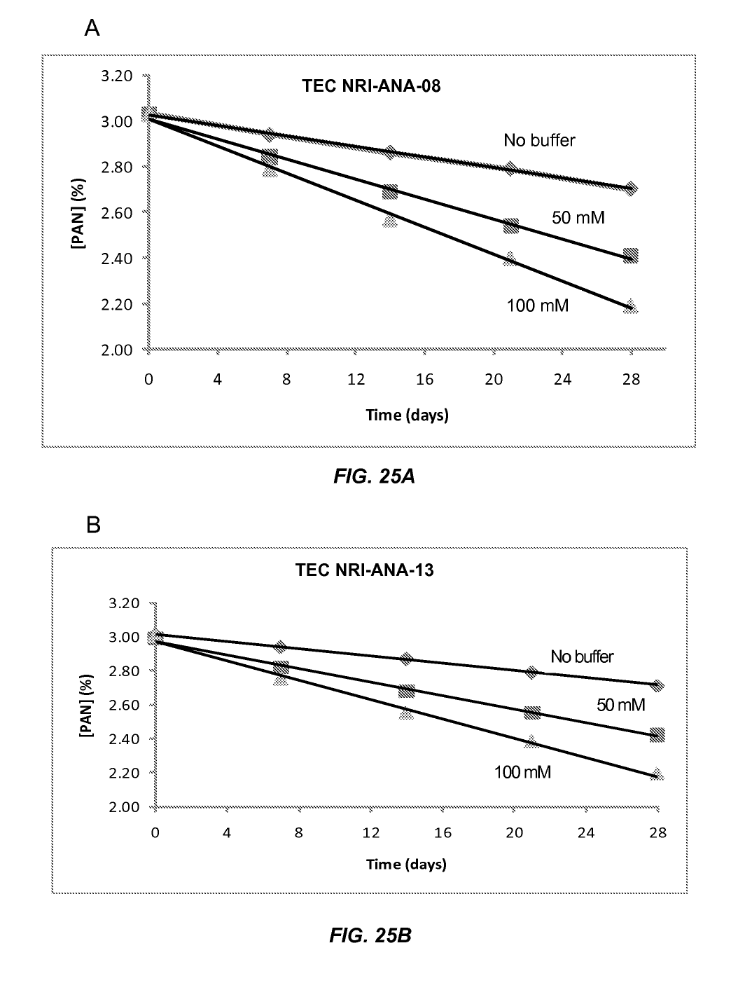

Buffering formulations NRI-ANA-08 and NRI-ANA-13 with citrate (50 or 100 mM) helped to reduce the change in pH but accelerated the degradation of PAN, in a concentration-dependent fashion in the pH range 3.5-4.5. See FIGS. 25A and 25B. This suggests that hydrolysis of TEC is catalyzed by citrate buffer at pH values less than 4.5.

The rate degradation of both PAN and TEC was also found to be lower in the formulations than predicted by the pH rate profiles, even after correction for buffer catalysis, perhaps because of the non-aqueous solvents in the formulation.

### Summary: Effects of 3-Aminopropanol

The initial pH values of the three formulations were consistent with levels of AMP present as an impurity in the panthenol. The NRI-ANA-14+0.0006% AMP formulation (analogous to formulation NRI-ANA-08) was pH 4.2 (vs. observed 4.1). The NRI-ANA-14+0.012% AMP formulation (analogous to formulation NRI-ANA-13) was pH 4.8 (vs observed 4.8). The control NRI-ANA-14+0.0% AMP formulation was pH 4.2.

However, addition of AMP to NRI-ANA-14 alone did not reduce the rate of degradation of TEC sufficiently to match the rate seen in NRI-ANA-08 and NRI-ANA-13. This may be attributed to the fact that PAN (and thus AMP arising from hydrolysis) is absent from NRI-ANA-14. The production of AMP via hydrolysis of PAN may be as important in controlling the pH of the formulation and thus reducing the rate of hydrolysis of TEC as the initial concentration of AMP.

### Example 22

### Effect of Formulations on the Permeation of Lidocaine from Abraded Cadaver Skin

This experiment investigated the permeation behavior of lidocaine from panthenol and panthenol-free formulations with pH-adjusted citrate buffer from abraded cadaver skin.

### Procedure: Tape Stripping

Cadaver skin pieces were placed on a cutting block with the stratum corneum side facing up. 3″-wide packing tape was used to abrade the skin and remove the stratum corneum. The tape was loosely applied to the skin surface to cover the entire area. Once the tape had been applied to the skin, the rubber pad of a pneumatic clamping chamber was placed on top of taped skin surface. The entire assembly was slid into the pneumatic clamping chamber. Once the assembly was properly aligned in the pneumatic clamping chamber, the pump was turned on until a clamping pressure of 12 psi was reached. The assembly was left to sit for about 10 seconds. The pneumatic bellows were then deflated by switching off the vacuum. Once the bellows were deflated, the assembly was slid out of the clamping chamber, and the rubber pad was removed from the skin surface. The tape was peeled off the skin in a gentle and uniform manner to remove a layer of stratum corneum cells. The process was repeated 10 times.

The rate of permeation of lidocaine was tested using Franz cells and included integrity testing with tritiated water. Lidocaine concentrations were analyzed by HPLC.

TABLE 24A
Composition of Test Formulations

| | Formulations | |
|---|---|---|
| | NRI-ANA-23 | NRI-ANA-8 |
| Ingredients | (F2) | (F1) |
| Percentage in | w/w % | |
| Lidocaine HCl monohydrate (USP)* | 10.67 | 10.67 |
| Triethyl Citrate (NF) | 2 | 2 |
| Diethylene Glygol Monoethyl Ether | 8.5 | 8.5 |
| (Transcutol) (NF) | | |
| Polysorbate (Tween) 20 (NF) | 8.1 | 8.1 |
| DL-Panthenol (USP) | 0 | 3 |
| Methyl Paraben (NF) | 0.1 | 0.1 |
| Propyl Paraben (NF) | 0.1 | 0.1 |
| 100 mM Citrate Buffer, pH 4.2 | 70.5 | |
| Purified Water (USP) | | 67.5 |

TABLE 24B
Permeation of Formulations Through Abraded Cadaver Skin
Accumulated Doses (μg/cm²)

| Time | Lidoderm ®_s | F1_s | F2_s |
|---|---|---|---|
| 2 hrs | 8.16 | 35.04 | 22.04 |
| 4 hrs | 29.16 | 58.14 | 37.32 |
| 6 hrs | 54.33 | 69.78 | 47.23 |
| 10 hrs | 109.54 | 87.80 | 65.11 |

F1_s: NRI-ANA-08 (formulation with panthenol) on tape stripped skin
F2_s: NRI-ANA-23 (formulation without panthenol) on tape stripped skin
Lidoderm_s: Lidoderm ® on tape stripped skin

TABLE 24C
Permeation Test Standard Error
Standard Error in Accumulated Doses (μg/cm²)

| Time | Lidoderm ®_s | F1_s | F2_s |
|---|---|---|---|
| 2 hrs | 1.16 | 8.61 | 6.49 |
| 4 hrs | 3.38 | 15.68 | 9.96 |
| 6 hrs | 5.87 | 18.09 | 11.97 |
| 10 hrs | 11.70 | 21.10 | 15.48 |

TABLE 24D
Enhancement Ratio Compared to Lidoderm ®
Permeation relative to Lidoderm ®

| Time | Lidoderm ®_s | F1_s | F2_s |
|---|---|---|---|
| 4 hrs ER | 1.00 | 1.99 | 1.28 |
| 6 hrs ER | 1.00 | 1.28 | 0.87 |
| 10 hrs ER | 1.00 | 0.80 | 0.59 |

### Results

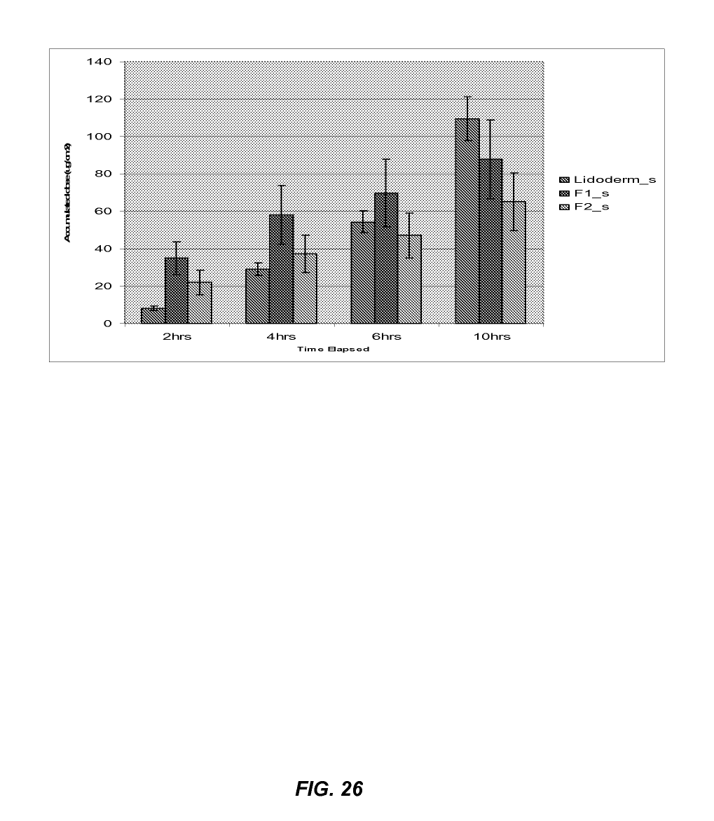

The results are shown in FIG. 26. Permeation of formulations appear to exhibit nearly similar trend to Lidoderm® up to 10 hours.

The formulation with panthenol appears to exhibit somewhat higher permeation than the panthenol-free formulation. This behavior appears to be similar to abraded porcine skin (see below).

### Example 23

### Permeation of Lidocaine Through Abraded and Intact Porcine Skin

Permeation behavior of lidocaine from panthenol and panthenol-free formulations from abraded and intact porcine skin.

### Procedure: Tape-Stripping

Porcine skin pieces were placed on a cutting block with the stratum corneum side facing up. 3″ wide packing tape was used to abraid the skin and remove the stratum corneum, the tape was loosely applied to the skin surface to cover the entire area. Once the tape has been applied to the skin, the rubber pad of a pneumatic clamping chamber was placed on top of taped skin surface. The entire assembly was slid into the pneumatic clamping chamber. Once the assembly was properly aligned in the pneumatic clamping chamber, the pump was turned on until a clamping pressure of 12 psi is reached. The assembly was left to sit for ˜10 seconds. The pneumatic bellows were then deflated by switching off the vacuum. Once the bellows were deflated, the assembly was slid out of the clamping chamber and the rubber pad was removed from the skin surface. The tape was peeled off the skin in a gentle and uniform manner to remove a layer of stratum corneum cells. The process was repeated 20 times.

The rate of permeation was tested using Franz cells and included integrity testing with tritiated water. The results were analyzed by HPLC.

TABLE 25A
Composition of Test Formulations

| | Formulations | |
|---|---|---|
| | NRI-ANA-23 | NRI-ANA-8 |
| Ingredients | (F2) | (F1) |
| Percentage in | w/w % | |
| Lidocaine HCl monohydrate (USP)* | 10.67 | 10.67 |
| Triethyl Citrate (NF) | 2 | 2 |
| Diethylene Glygol Monoethyl Ether | 8.5 | 8.5 |
| (Transcutol) (NF) | | |
| Polysorbate (Tween) 20 (NF) | 8.1 | 8.1 |
| DL-Panthenol (USP) | 0 | 3 |
| Methyl Paraben (NF) | 0.1 | 0.1 |
| Propyl Paraben (NF) | 0.1 | 0.1 |
| 100 mM Citrate Buffer, pH 4.2 | 70.5 | |
| Purified Water (USP) | | 67.5 |

TABLE 25B
Permeation of Formulations Through Intact Porcine Skin
Accumulated Doses (μg/cm²)

| Formula | Lidoderm ® | F1 | F2 |
|---|---|---|---|
| 2 hrs | 0.00 | 1.83 | 1.93 |
| 4 hrs | 1.07 | 4.97 | 4.35 |
| 6 hrs | 3.99 | 8.85 | 8.34 |
| 9 hrs | 6.68 | 13.22 | 12.49 |
| 21 hrs | 19.51 | 24.10 | 20.79 |
| 21 hrs ER | 1.00 | 1.24 | 1.07 |
| Dosing (ul) | | 3.00 | 3.00 |

F1: NRI-ANA-08 (formulation with panthenol)
F2: NRI-ANA-23 (formulation without panthenol)

TABLE 25C
Permeation Test (Intact Porcine Skin) Standard Error
Standard Error in Accumulated Doses (μg/cm²)

| Time | Lidoderm ® | F1 | F2 |
|---|---|---|---|
| 2 hrs | 0.00 | 0.88 | 0.68 |
| 4 hrs | 0.61 | 1.99 | 1.56 |
| 6 hrs | 1.27 | 2.59 | 2.38 |
| 9 hrs | 2.18 | 2.99 | 2.72 |
| 21 hrs | 7.12 | 4.35 | 4.62 |

TABLE 25D
Permeation of Formulations Through Abraded Porcine Skin
Accumulated does in (ug/cm2)

| Formula | Lidoderm_s | F1_s | F2_s |
|---|---|---|---|
| 2 hrs | 1.91 | 13.34 | 9.11 |
| 4 hrs | 6.56 | 30.71 | 20.89 |
| 6 hrs | 13.89 | 43.56 | 29.94 |
| 10 hrs | 23.36 | 53.91 | 37.34 |
| 21 hrs | 60.45 | 74.77 | 58.15 |
| 21 hrs ER | 1.00 | 1.24 | 0.96 |
| Dosing (ul) | | 3.00 | 3.00 |

F1_s: NRI-ANA-08 (formulation with panthenol) on tape stripped skin
F2_s: NRI-ANA-23 (formulation without panthenol) on tape stripped skin
Lidoderm_s: Lidoderm ® on tape stripped skin

TABLE 25E
Permeation Test (Abraded Porcine Skin) Standard Error
Standard Error in Accumulated Doses (μg/cm²)

| Time | Lidoderm ®_s | F1_s | F2_s |
|---|---|---|---|
| 2 hrs | 1.01 | 3.95 | 3.39 |
| 4 hrs | 2.85 | 7.74 | 7.81 |
| 6 hrs | 4.81 | 11.63 | 11.42 |
| 10 hrs | 7.74 | 14.33 | 13.19 |
| 21 hrs | 17.48 | 18.76 | 17.12 |

### Results

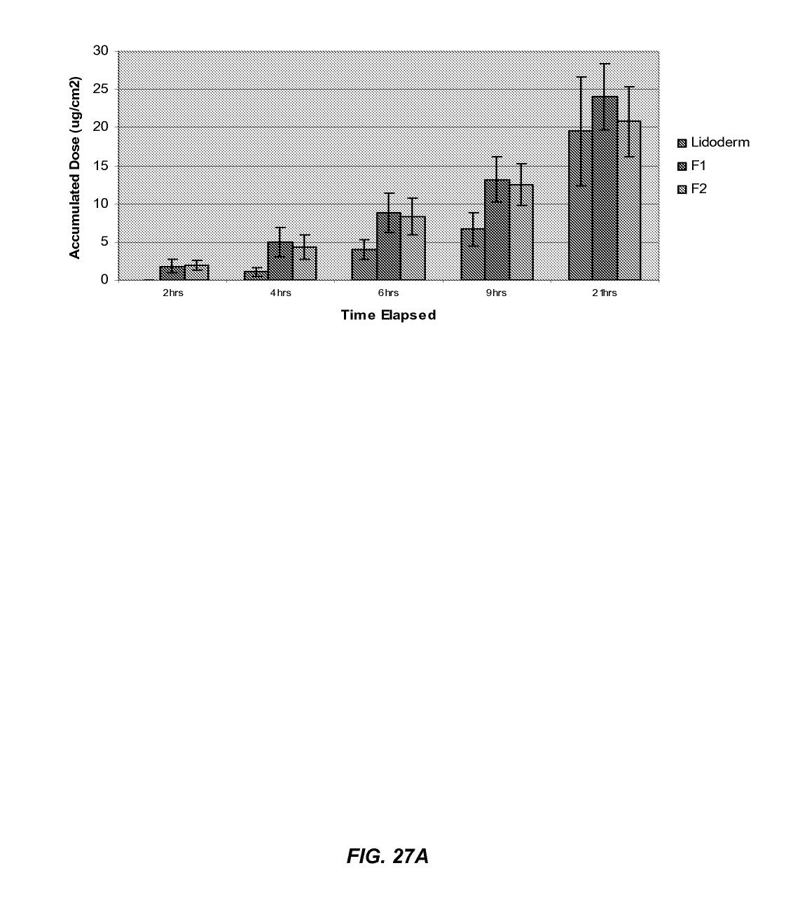

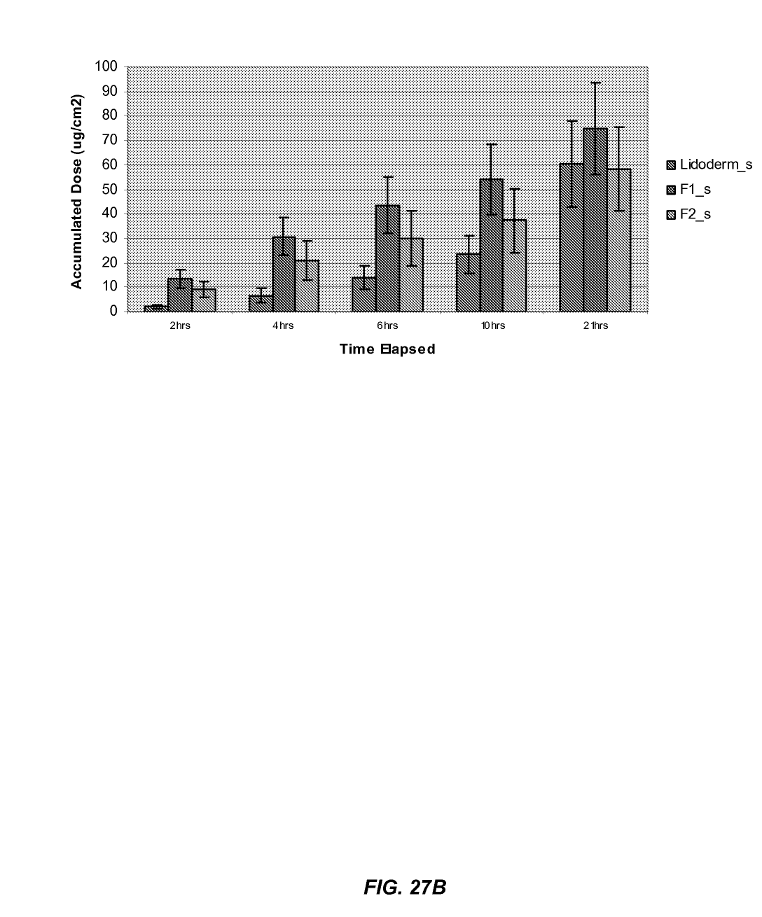

The results are shown in FIGS. 27A-B. Permeation of formulations appear to exhibit nearly similar trends to Lidoderm® except that at early hours no permeation from the Lidoderm® patch was observed. The formulation with panthenol (F1, F1_s) appears to exhibit somewhat higher permeation than to the panthenol-free formulation (F2, F2_s).

### Example 24

### Skin Sensory Testing Following Application of Placebo Formulation

The objective of the study was to compare the skin sensory perception of stinging from a placebo topical formulation when applied to slightly abraded skin in a randomized, single-blind, single-dose exposure study. Ten subjects (one male, nine females) who met the study criteria were enrolled. All enrolled subjects completed the study.

Procedure for Measuring Skin Barrier Function with TEWL

Transepidermal water loss (TEWL) was measured with a Dermalab Evaporimeter (Cortex Technology, Denmark). Each measurement consisted of a 60-second collection period (40 seconds equilibration and 20 seconds averaged readings). Instrument assessments were conducted in a room maintained at 18-25° C. and 30-40% relative humidity. Subjects were required to equilibrate with the ambient environmental conditions of the measurement room for at least 30 minutes prior to the pre-wounding TEWL assessment of the first site. Temperature and humidity data were recorded.

One measurement was taken from each test site prior to tape stripping (values lower than 10 g/m²/h were targeted). If any site had a pre-wounding TEWL 10 g/m²/h, the subject was asked to rest quietly for an additional 15 minutes, and the TEWL was repeated for those particular sites. If the pre-wounding TEWL remained 10 g/m²/h for any test site, the subject was not eligible to continue in the study.

A TEWL measurement was taken from each test site post-tape-stripping (see below) to confirm that the barrier function had been compromised. TEWL values post-stripping had to be g/m²/h, in order to maintain consistent barrier damage among the wounded sites. Tape stripping and TEWL measurements were repeated until this level is reached.

### Tape-Stripping

Blenderm™ surgical tape (3 M™) was used for tape stripping. Test sites with dimensions of 2.5 cm×4 cm were marked on the volar forearm of the subjects. Gloves or finger cots were worn during tape stripping to avoid wound contact. Gloves were changed between subjects. Skin sites underwent a tape-stripping procedure to create a superficial wound down to the glistening layer and to compromise the skin barrier.

Strips of Blenderm™ tape were cut to approximately 7.0×2.5 cm. A tape strip was placed on the test site, pressed down, rubbed firmly within site marks, and removed with a strong and quick stroke. The tape was discarded. The stripping was repeated using other tape strips, in alternate directions, until a clear glistening layer could be visualized or after 39 times (40 strips total), whichever came first. The number of stripping steps necessary to reach the glistening layer varied among subjects.

After tape stripping, a TEWL measurement was taken. The target TEWL value after tape stripping was ≧30 g/m²/h. If TEWL was <30 g/m²/h, an additional 10 tape stripping steps were taken (or less, if glistening was visualized), and then the TEWL was measured again. This process was repeated until a TEWL value≧30 g/m²/h was reached.

### Treatment Assignments

There were a total of 3 sites on each arm for a total of 6 test sites. Of these, four sites were dosed with one of the four placebo test formulations. The remaining two sites were dosed with either the negative control (water) or the positive control (70% isopropyl alcohol). Treatment assignments to the test sites were randomized, and the order of application of the test articles to their assigned sites was randomized. The subjects were blinded with respect to the treatment assignments. The randomization schedule was provided by Hill Top Research.

### Test Article Application

All applied doses were 5.0 μL/cm² of formulation to a 10 cm² site. The application was made using an Eppendorf™ repeat dose pipette (or equivalent) set to deliver 50 μL, and was evenly spread and gently distributed throughout the test site with a glass rod. The applied doses remained on the skin for at least 10 minutes or until the last assessment on the last site was collected.

### Sensory Assessment

Immediately after dosing and at 2, 5, and 10 minutes after the dose application of each test article, the subject was asked the question: “Do you feel any stinging, pain, or discomfort sensation at this site?” The subject was requested to respond using a 100-mm visual analogue scale anchored at one end with ‘None’ (equal to 0 mm) and the other end with ‘Severe’ (equal to 100 mm). The subject was instructed to place a single vertical line on the scale that best indicated the degree of the stinging/pain discomfort. Using calipers, the study technician measured the distance from zero (i.e. ‘None’) to the mark on the scale made by the subject, and recorded this value for each post-dosing assessment for each test site.

At the end of the 10 minutes, the subject was asked to read and answer the following question for each test article: “Describe any sensations (bad or good) that you experienced following application of this product.” The subject recorded his/her answer.

### Surface Wash

After the 10 minute assessment for each site, the site was rinsed with water to remove residual test article. Sites not yet dosed were protected from the water rinse of an adjacent site. If stinging persisted after rinsing, the subject rested quietly until there was no stinging sensation, and only then was the next test site tape-stripped and dosed.

If a site presented intolerable stinging within the 10 minute assessment period, it was rinsed with water, and the last score was carried forward through the remaining assessment times.

### Statistical Analysis

Descriptive statistics (mean, standard deviation, median, minimum, maximum) were provided for the responses to the stinging, pain and discomfort sensory question, individually and overall, for each time point post-dosing, and for the maximum stinging, pain and discomfort responses, individually and overall, for each of the test articles. The data used in the statistical analysis were the maximum score assigned for each treatment for stinging, pain and discomfort, each sensation individually, and for the maximum score overall for any sensation. Wilcoxon's Signed Rank Test was used to compare the maximum response for each of the placebo topical formulations and the positive control to the maximum response for the negative control.

### Results

Test Articles: Treatment D was a test (placebo) formulation of the composition shown in Table 26A. Treatment E was water (a negative control). Treatment F was 70% isopropyl alcohol (a positive control).

TABLE 26A
Composition of Test Formulation (Treatment D)

| | Formulation Code |
|---|---|
| | D |
| Ingredient | (%, wt/wt) |
| Panthenol (racemic) | 3.0 |
| Triethyl citrate | 2.0 |
| Transcutol | 8.5 |
| Tween 20 | 8.1 |
| Methyl paraben | 0.1 |
| Propyl paraben | 0.1 |
| Water | 78.2 (q.s.) |

TABLE 26B
Subjective Stinging Scores in Comparison to Control E

| | | | | Mean | | |
|---|---|---|---|---|---|---|
| | | | | Control | Mean | |
| | | | Mean | Score | Difference | Signed |
| | | | Treated | (HTR | from | Rank p- |
| Treatment | Visit | n | Score | Code E) | Control (E) | value¹ |
| D | Immediate | 10 | 1.26 | 2.45 | | |
| | 2 Minute | 10 | 1.85 | 3.91 | | |
| | 5 Minute | 10 | 1.47 | 3.01 | | |
| | 10 Minute | 10 | 1.59 | 1.70 | | |
| | Maximum | 10 | 2.32 | 4.80 | −2.48 | 0.1640 |
| F | Immediate | 10 | 10.84 | 2.45 | | |
| | 2 Minute | 10 | 3.31 | 3.91 | | |
| | 5 Minute | 10 | 1.87 | 3.01 | | |
| | 10 Minute | 10 | 1.00 | 1.70 | | |
| | Maximum | 10 | 12.13 | 4.80 | 7.33 | 0.2753 |

¹No significant difference between the treated sites and negative control site

TABLE 26C
Subjective Pain Scores in Comparison to Control E

| | | | | Mean | | |
|---|---|---|---|---|---|---|
| | | | | Control | Mean | |
| | | | Mean | Score | Difference | Signed |
| | | | Treated | (HTR | from | Rank p- |
| Treatment | Visit | n | Score | Code E) | Control (E) | value¹ |
| D | Immediate | 10 | 1.51 | 1.17 | | |
| | 2 Minute | 10 | 1.63 | 2.13 | | |
| | 5 Minute | 10 | 1.70 | 2.70 | | |
| | 10 Minute | 10 | 1.41 | 1.82 | | |
| | Maximum | 10 | 2.07 | 2.87 | −0.80 | >0.5000 |
| F | Immediate | 10 | 3.91 | 1.17 | | |
| | 2 Minute | 10 | 2.77 | 2.13 | | |
| | 5 Minute | 10 | 1.96 | 2.70 | | |
| | 10 Minute | 10 | 1.04 | 1.82 | | |
| | Maximum | 10 | 5.10 | 2.87 | 2.24 | 0.1289 |

¹No significant difference between the treated sites and negative control site

TABLE 26D
Subjective Discomfort Scores in Comparison to Control E

| | | | | Mean | | |
|---|---|---|---|---|---|---|
| | | | | Control | Mean | |
| | | | Mean | Score | Difference | Signed |
| | | | Treated | (HTR | from | Rank p- |
| Treatment | Visit | n | Score | Code E) | Control (E) | value¹ |
| D | Immediate | 10 | 1.59 | 1.51 | | |
| | 2 Minute | 10 | 1.96 | 2.43 | | |
| | 5 Minute | 10 | 1.78 | 2.89 | | |
| | 10 Minute | 10 | 1.89 | 2.07 | | |
| | Maximum | 10 | 2.42 | 3.07 | −0.65 | >0.5000 |
| F | Immediate | 10 | 4.85 | 1.51 | | |
| | 2 Minute | 10 | 2.65 | 2.43 | | |
| | 5 Minute | 10 | 2.09 | 2.89 | | |
| | 10 Minute | 10 | 1.44 | 2.07 | | |
| | Maximum | 10 | 5.95 | 3.07 | 2.88 | 0.1054 |

¹No significant difference between the treated sites and negative control site

TABLE 26E
Overall Subjective Scores in Comparison to Control E

| | | | | Mean | | Signed |
|---|---|---|---|---|---|---|
| | | | | Control | Mean | Rank |
| | | | Mean | Score | Difference | Patent |
| | | | Treated | (HTR | from | No.- |
| Treatment | Visit | n | Score | Code E) | Control (E) | value¹ |
| D | Immediate | 10 | 1.45 | 1.75 | | |
| | 2 Minute | 10 | 1.81 | 2.82 | | |
| | 5 Minute | 10 | 1.69 | 2.59 | | |
| | 10 Minute | 10 | 1.63 | 1.86 | | |
| | Maximum | 10 | 2.63 | 4.93 | −2.31 | 0.2031 |
| F | Immediate | 10 | 6.53 | 1.71 | | |
| | 2 Minute | 10 | 2.91 | 2.82 | | |
| | 5 Minute | 10 | 1.76 | 2.59 | | |
| | 10 Minute | 10 | 1.16 | 1.86 | | |
| | Maximum | 10 | 12.49 | 4.93 | 7.56 | 0.2324 |

¹No significant difference between the treated site and negative control.site

TABLE 26F
Summary Of Mean (SD) Maximum Subjective Scores, mm

| Treatment | Stinging | Pain | Discomfort | Overall |
|---|---|---|---|---|
| D | 2.32 (2.36) | 2.07 (2.05) | 2.42 (2.22) | 2.63 (2.40) |
| E | 4.80 (6.73) | 2.87 (2.98) | 3.07 (3.33) | 4.93 (6.65) |
| F | 12.13 (19.06) | 5.10 (4.78) | 5.95 (6.08) | 12.49 (18.91) |

TABLE 26G
Distribution of Score and Maximum Score

| | No. Subjects with Score: | | | Maximum VAS |
|---|---|---|---|---|
| Treatment | >5 mm | >10 mm | >20 mm | (any subject), mm |
| D | 2 | 0 | 0 | 7.06 |
| E | 3 | 2 | 0 | 19.70 |
| F | 6 | 3 | 1 | 64.16 |

Results: The mean maximum stinging score, pain score, discomfort score and maximum score overall for any sensation for Treatment D were very low (<2.6 mm on VAS) and were lower than the maximum scores, respectively, for the negative control, Treatment E.

### Example 25

### Acute Dermal Irritation/Corrosion Evaluation in Rabbits

The potential skin irritation/corrosion properties of the test article formulation will be assessed via conduct of a “Primary Skin Irritation Study in Rabbits” in accordance with the Organization of Economic Co-operation and Development (OECD) 404 guidance (revision 1992) (OECD (2004). Guideline for the Testing of Chemicals, No. 404: Acute Dermal Irritation/Corrosion. 13 pp. Paris, France: OECD).

“Skin irritation” as used in this example refers to the production of reversible damage to the skin following the application of a test substance for up to 4 hours.

“Dermal corrosion” as used in this example refers to the production of irreversible damage of the skin (visible necrosis [through the epidermis and into the dermis] typified by ulcers, bleeding, bloody scabs, skin discoloration, complete areas of alopecia, and scarring).

In brief, an initial pilot test will be conducted using one New Zealand White (NZW) rabbit to assess corrosive potential. If the test formulation is not shown to be corrosive, a confirmatory test will be conducted using a single group of 2-3 NZW rabbits of a single gender to assess irritation potential. In each test (pilot and confirmatory), the rabbit(s) will receive a single 4 hour semi-occluded topical dose administration of the test formulation. An untreated skin site will serve as the control. The degree of irritation/corrosion will be assessed according to the dermal scoring method for erythema/edema described in the OECD 404 guidance (OECD (2004). Guideline for the Testing of Chemicals, No. 404: Acute Dermal Irritation/Corrosion. 13 pp. Paris, France: OECD). Dermal scoring will be conducted for up to 14 days following exposure in order to determine the reversibility of effects.

The details of the test procedure are as follows:

### Animal Care

Rabbits will be individually housed and maintained in accordance with Good Laboratory Practice (GLP) regulations (The Nonclinical Laboratory Studies Good Laboratory Practice Regulations issued by the U.S. Food and Drug Administration (FDA), Title 21 of the Code of Federal Regulations, Part 58; effective Jun. 20, 1979; The OECD Principles on Good Laboratory Practice (C[97]186/Final; effective 1997); The Japanese Good Laboratory Practice Standards for Safety Studies on Drugs (Ordinance No. 21 of the Pharmaceutical Affairs Bureau, Ministry of Health, Labor and Welfare [MHLW], Japan; effective Apr. 1, 1997)). Animals showing continuing signs of severe distress and/or pain at any stage of the study will be humanely killed and the substance assessed accordingly.

### Irritation Test

Approximately 24 hours pre-dose, two skin sites on the dorsal trunk of each rabbit will be prepared by careful clipping of fur and sites will be inspected to ensure healthy, intact skin.

A dose of 0.5 mL of undiluted test article formulation will be applied to the prepared skin site (˜6 cm²) and covered with a gauze patch which will be held in place with non-irritating tape for 4 hours. Access by the rabbit to the patch will be prevented by means of a collar.

At the completion of the 4 hour exposure period, the patch will be discarded and the residual test formulation will be carefully removed from the skin using clear water.

### Irritation/Corrosion Results

The test site will be examined and scored immediately following patch removal (initial test only), then at 60 minutes and 24, 48 and 72 hours. If irritation is observed, daily dermal observations/scoring will be conducted for up to 14 days after dosing.

In addition to dermal scoring, all toxic effects (defatting of skin, clinical signs, body weights) will be recorded and reported. If warranted by dermal signs, histopathological evaluation of the skin site may be performed.

Dermal irritation scores will be tabulated and evaluated in conjunction with the nature and severity of lesions, and the status of reversibility. The irritation potential of the formulation will be categorized as non-irritating or irritating based on the dermal response. If responses such as alopecia (limited area), hyperkeratosis, hyperplasia and scaling, persist to the end of the 14 day observation period, the test formulation will be classified as an irritant even in the absence of an acute irritation response (erythema/edema). Data will be collated and discussed in the form of a final study report.

All publications, patents and patent applications referred to herein are incorporated by reference in their entirety to the same extent as if each individual publication, patent or patent application was specifically and individually indicated to be incorporated by reference in its entirety.

1. A topical composition, said composition comprising:
a) a topically acting anesthetic active ingredient;
b) an ester selected from the group consisting of a citric acid ester and ethyl acetate;
c) a non-ionic surfactant;
d) a polar solvent; and
e) water or an aqueous buffer.

2. (canceled)

3. (canceled)

4. The composition of claim 1, wherein said anesthetic active ingredient is in a subanesthetic amount.

5. The composition of claim 1, wherein said anesthetic active ingredient is in the amount of about 0.1% to about 20% w/w.

6. The composition of claim 5, wherein said anesthetic active ingredient is in the amount of about 6% to about 13% w/w.

7. The composition of claim 1, wherein said anesthetic active ingredient is selected from the group consisting of tetracaine, lidocaine, prilocalne, benzocaine, bupivacaine, mepivacaine, dibucaine, etidocaine, butacaine, cyclomethycaine, hexylcaine, proparacaine, lopivacaine and pharmaceutically acceptable salts thereof.

8. The composition of claim 7, wherein said anesthetic active ingredient is selected from the group consisting of lidocaine hydrochloride and lidocaine base.

9. The composition of claim 1, wherein said composition is substantially free of a lower alkanol.

10. (canceled)

11. The composition of claim 1, wherein said ester is a citric acid ester.

12. (canceled)

13. The composition of claim 11, wherein said citric acid ester is a member selected from the group consisting of triethyl citrate, acetyl triethyl citrate, tributyl citrate, acetyl tributyl citrate and a combination thereof.

14. The composition of claim 13, wherein said citric acid ester is triethyl citrate.

15. (canceled)

16. (canceled)

17. (canceled)

18. The composition of claim 1, wherein said non-ionic surfactant is a member selected from the group consisting of sorbitan monolaurate, sorbitan monopalmitate, sorbitan monostearate, sorbitan sesquistearate, polyoxyethylene sorbitan monolaurate, polyoxyethylene monopalmitate, polyoxyethylene sorbitan monostearate, polyoxyethylene sorbitan tristearate, polyoxyethylene sorbitan monooleate, polyoxyethylene sorbitan trioleate, polyoxyethylene sorbitol monolaurate, polyoxyethylene sorbitol hexastearate, polyoxyethylene sorbitol tetraoleate, polyoxyethylene lauryl ester, polyoxyethylene stearyl ester, polyoxyethylene oleyl ester, polyoxyethylene lauryl ether, polyoxyethylene cetyl ether, polyoxyethylene stearyl ether, polyoxyethylene oleyl ether, polyoxyethylene hexadecyl ether, propylene glycol monostearate, polyoxypropylene, polyoxyethylene cetyl ether and a combination thereof.

19. The composition of claim 18, wherein said non-ionic surfactant is a member selected from the group consisting of polyoxyethylene (20) sorbitan monolaurate and polyoxyethylene (20) sorbitan monooleate.

20. (canceled)

21. The composition of claim 1, wherein said polar solvent is a member selected from the group consisting of a diol, a triol, a polyol, transcutol, a low-weight PEG, and panthenol.

22. (canceled)

23. (canceled)

24. (canceled)

25. (canceled)

26. The composition of claim 1, wherein said polar solvent is diethylene glycol monoethyl ether (transcutol).

27. (canceled)

28. (canceled)

29. (canceled)

30. (canceled)

31. (canceled)

32. The composition of claim 1, further comprising a preservative selected from the group consisting of propyl paraben, methyl paraben or a combination thereof.

33. (canceled)

34. (canceled)

35. (canceled)

36. (canceled)

37. The composition of claim 1, wherein the buffer is a citrate buffer.

38. The composition of claim 37, wherein the concentration of the buffer ranges from about 1 mM to about 500 mM.

39. (canceled)

40. (canceled)

41. (canceled)

42. (canceled)

43. (canceled)

44. (canceled)

45. (canceled)

46. (canceled)

47. (canceled)

48. (canceled)

49. A method for alleviating pain, said method comprising:
applying to an affected area a topical composition, said composition comprising:
a) a topically acting anesthetic active ingredient;
b) an ester selected from the group consisting of a citric acid ester and ethyl acetate;
c) a non-ionic surfactant;
d) a polar solvent; and
e) water or an aqueous buffer, thereby alleviating pain.

50. (canceled)

51. (canceled)

52. The method of claim 49, wherein the method of application of the composition is by spraying.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. The method of claim 49, wherein said pain is experienced by a human subject afflicted by acute herpes zoster.

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)
